SEQUENCE LISTING (3 sequences)

SEQ ID NO: 1 - protein, 310 aa, Homo sapiens
KFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHS

SEQ ID NO: 2 - protein, 250 aa, Homo sapiens [sequence omitted]

SEQ ID NO: 3 - protein, 76 aa, Homo sapiens
HPLGSPGSASDLETSGLQEQRNHLQGKLSELQVEQTSLEPLQESPRPTGVWKSREVATEGIRGHRKMVLYTLRAPR

(19) United States
(12) Patent Application Publication
(10) Pub. No.: US 2024/0361336 A1
(43) Pub. Date: Oct. 31, 2024

(54) NEWBORN SCREENING FOR CONGENITAL HEART DEFECT USING CARDIOVASCULAR BIOMARKERS IN DRIED BLOOD SAMPLES

(71) Applicant: Wallac Oy, Turku (FI)

(72) Inventor: Mikko Sairanen, Turku (FI)

(21) Appl. No.: 18/647,364

(22) Filed: Apr. 26, 2024

Related U.S. Application Data
(60) Provisional application No. 63/462,804, filed on Apr. 28, 2023.

(51) Int. Cl. G01N 33/68; G16H 50/20

(52) U.S. Cl. CPC G01N 33/6893; G16H 50/20; G01N 2333/4703; G01N 2333/58

(57) ABSTRACT

Methods of detection of congenital heart defect (CHD) in a newborn human patient, are provided according to aspects of the present disclosure which include: performing an assay for one or more biomarkers of CHD selected from the group consisting of: soluble Suppression of Tumorigenicity 2 protein (sST2), and Galectin-3 in a dried blood sample obtained from the newborn human patient, thereby determining a level of one or both biomarkers present in the dried blood sample. The determined level of the biomarker(s) present in the dried blood sample is compared with a standard representing a normal newborn control without CHD, thereby determining whether the determined level is increased or decreased compared to the standard, wherein an increased level of one or both biomarkers present in the dried blood sample, compared with a standard representing a normal newborn control without congenital heart defect, indicates detection of CHD in the newborn human patient.

### REFERENCE TO RELATED APPLICATION

This application claims priority from U.S. Provisional Patent Application Ser. No. 63/462,804, filed Apr. 28, 2023, the entire content of which is incorporated herein by reference.

### SEQUENCE LISTING

The instant application contains a Sequence Listing which has been submitted electronically as a file in XML format and is hereby incorporated by reference in its entirety. Said XML format file, created on Apr. 25, 2024, is named 16NEN28902PA.xml and is 4,068 bytes in size.

### FIELD OF THE INVENTION

Generally described, aspects of the present disclosure relate to methods, kits, and computer-implemented methods for detection of congenital heart defect (CHD). According to specific aspects of the present disclosure, methods, kits, and computer-implemented methods are described relating to newborn screening for congenital heart defect using cardiovascular biomarkers in dried blood samples.

### BACKGROUND OF THE INVENTION

Currently, routine clinical screening for congenital heart defect (CHD) in newborns is limited to use of pulse oximetry (POX), a technique which misses some heart defects which can be fatal if not detected early enough. Thus, there is a continuing need for methods for improved detection of CHD in newborns.

### SUMMARY OF THE INVENTION

Methods of detection of congenital heart defect (CHD) in a newborn human patient are provided according to aspects of the present disclosure which include: performing an assay for one or both biomarkers of CHD selected from the group consisting of: soluble Suppression of Tumorigenicity 2 protein (sST2), and Galectin-3, in a dried blood sample obtained from the newborn human patient, thereby determining a level of the one or more biomarkers present in the dried blood sample. The determined level of the one or more biomarkers present in the dried blood sample is compared with a standard representing a normal newborn control without CHD, thereby determining whether the determined level is increased or decreased compared to the standard, wherein an increased level of the one or more biomarkers present in the dried blood sample compared with a standard representing a normal newborn control without congenital heart defect indicates detection of CHD in the newborn human patient.

Methods of detection of congenital heart defect (CHD) in a newborn human patient are provided according to aspects of the present disclosure which include: performing an assay for two or more biomarkers of CHD selected from the group consisting of: soluble Suppression of Tumorigenicity 2 protein (sST2), N-terminal pro b-type natriuretic peptide (NT-proBNP), and Galectin-3, in a dried blood sample obtained from the newborn human patient, thereby determining a level of the two or more biomarkers present in the dried blood sample. The determined level of the two or more biomarkers present in the dried blood sample is compared with a standard representing a normal newborn control without CHD, thereby determining whether the determined level is increased or decreased compared to the standard, wherein an increased level of the two or more biomarkers present in the dried blood sample compared with a standard representing a normal newborn control without congenital heart defect indicates detection of CHD in the newborn human patient.

Methods of detection of congenital heart defect (CHD) in a newborn human patient are provided according to aspects of the present disclosure which include: performing an assay for: 1) sST2 and NT-proBNP; or 2) sST2 and Galectin-3; or 3) NT-proBNP and Galectin-3, or 4) all of sST2, NT-proBNP and Galectin-3, in a dried blood sample obtained from the newborn human patient, thereby determining a level of the two or more biomarkers present in the dried blood sample. The determined level of the two or more biomarkers present in the dried blood sample is compared with a standard representing a normal newborn control without CHD, thereby determining whether the determined level is increased or decreased compared to the standard, wherein an increased level of the two or more biomarkers present in the dried blood sample compared with a standard representing a normal newborn control without congenital heart defect indicates detection of CHD in the newborn human patient.

Methods of detection of congenital heart defect (CHD) in a newborn human patient are provided according to aspects of the present disclosure which include: performing an immunoassay for one or both biomarkers of CHD selected from the group consisting of: soluble Suppression of Tumorigenicity 2 protein (sST2), and Galectin-3, in a dried blood sample obtained from the newborn human patient, thereby determining a level of the one or more biomarkers present in the dried blood sample. The determined level of the one or more biomarkers present in the dried blood sample is compared with a standard representing a normal newborn control without CHD, thereby determining whether the determined level is increased or decreased compared to the standard, wherein an increased level of the one or more biomarkers present in the dried blood sample compared with a standard representing a normal newborn control without congenital heart defect indicates detection of CHD in the newborn human patient.

Methods of detection of congenital heart defect (CHD) in a newborn human patient are provided according to aspects of the present disclosure which include: performing an immunoassay for two or more biomarkers of CHD selected from the group consisting of: soluble Suppression of Tumorigenicity 2 protein (sST2), N-terminal pro b-type natriuretic peptide (NT-proBNP), and Galectin-3, in a dried blood sample obtained from the newborn human patient, thereby determining a level of the two or more biomarkers present in the dried blood sample. The determined level of the two or more biomarkers present in the dried blood sample is compared with a standard representing a normal newborn control without CHD, thereby determining whether the determined level is increased or decreased compared to the standard, wherein an increased level of the two or more biomarkers present in the dried blood sample compared with a standard representing a normal newborn control without congenital heart defect indicates detection of CHD in the newborn human patient.

Methods of detection of congenital heart defect (CHD) in a newborn human patient are provided according to aspects of the present disclosure which include: performing an immunoassay for: 1) sST2 and NT-proBNP; or 2) sST2 and Galectin-3; or 3) NT-proBNP and Galectin-3, or 4) all of sST2, NT-proBNP and Galectin-3, in a dried blood sample obtained from the newborn human patient, thereby determining a level of the two or more biomarkers present in the dried blood sample. The determined level of the two or more biomarkers present in the dried blood sample is compared with a standard representing a normal newborn control without CHD, thereby determining whether the determined level is increased or decreased compared to the standard, wherein an increased level of the two or more biomarkers present in the dried blood sample compared with a standard representing a normal newborn control without congenital heart defect indicates detection of CHD in the newborn human patient.

Optionally, the blood sample is obtained from the newborn human patient: on the day of birth, one day after birth, two days after birth, three days after birth, four days after birth, five days after birth, six days after birth, one week after birth, or later.

According to aspects of the present disclosure, the method further includes assessment of the newborn human patient using pulse oximetry (POX).

According to aspects of the present disclosure, the method further includes assessment of one or more physical characteristics of the newborn human patient such that the assay result is normalized with respect to the physical characteristic and is compared to a standard normalized to the same physical characteristic. According to aspects of the present disclosure, the method further includes assessment of one or more physiological characteristics of the newborn human patient such that the assay result is normalized with respect to the physiological characteristic and is compared to a standard normalized to the same physical characteristic.

According to aspects of the present disclosure, the assay result is normalized with respect to one or more of: birth weight, gestational age, sex of newborn, and day of testing after birth in normal (non-CHD) newborn human patients and is compared to a standard normalized to the same characteristic or characteristics.

Kits for detection of CHD in a newborn human patient, are provided according to aspects of the present disclosure, which include one or more reagents for performing an assay for one or both biomarkers of CHD selected from the group consisting of: soluble Suppression of Tumorigenicity 2 protein (sST2), and Galectin-3 in a dried blood sample obtained from the newborn human patient, to determine a level of the one or both biomarkers present in the dried blood sample.

Kits for detection of CHD in a newborn human patient, are provided according to aspects of the present disclosure, which include one or more antibodies specific for sST2, and/or Galectin-3.

Kits for detection of CHD in a newborn human patient, are provided according to aspects of the present disclosure, which include: 1) at least one antibody specific for sST2 and at least one antibody specific for NT-proBNP; or 2) at least one antibody specific for sST2 and at least one antibody specific for Galectin-3; or 3) at least one antibody specific for NT-proBNP and at least one antibody specific for Galectin-3, or 4) at least one antibody specific for sST2, at least one antibody specific for NT-proBNP, and at least one antibody specific for Galectin-3.

Kits for detection of CHD in a newborn human patient, are provided according to aspects of the present disclosure, which include: 1) at least one antibody specific for sST2 and at least one antibody specific for NT-proBNP; or 2) at least one antibody specific for sST2 and at least one antibody specific for Galectin-3; or 3) at least one antibody specific for NT-proBNP and at least one antibody specific for Galectin-3, or 4) at least one antibody specific for sST2, at least one antibody specific for NT-proBNP, and at least one antibody specific for Galectin; and one or more reagents or devices for one or more of: collection, drying, transport, and storage, of a blood sample from a newborn human patient.

Computer-implemented methods of detection of CHD in a newborn human patient are provided according to aspects of the present disclosure which include: providing a determined level of one or more biomarkers selected from the group consisting of: sST2, and Galectin-3 present in a dried blood sample of the newborn human patient; responsive to the determined level, assigning a score to the newborn human patient representative of detection of CHD by algorithmically comparing the determined level(s) to a standard or standards representing one or more normal newborn controls without congenital heart defect, thereby detecting congenital heart defect in the newborn human patient.

Computer-implemented methods of detection of CHD in a newborn human patient are provided according to aspects of the present disclosure which include: providing a determined level of: 1) sST2 and NT-proBNP; or 2) sST2 and Galectin-3; or 3) NT-proBNP and Galectin-3, or 4) all of sST2, NT-proBNP and Galectin-3, present in a dried blood sample of the newborn human patient; responsive to the determined level, assigning a score to the newborn human patient representative of detection of CHD by algorithmically comparing the determined level(s) to a standard or standards representing one or more normal newborn controls without congenital heart defect, thereby detecting congenital heart defect in the newborn human patient.

Computer-implemented methods of detection of CHD in a newborn human patient are provided according to aspects of the present disclosure which include: providing a determined level of one or more biomarkers selected from the group consisting of: sST2, and Galectin-3 present in a dried blood sample of the newborn human patient; responsive to the determined level, assigning a score to the newborn human patient representative of detection of CHD by algorithmically comparing the determined level(s) to a standard or standards representing one or more normal newborn controls without congenital heart defect, thereby detecting congenital heart defect in the newborn human patient. According to aspects of the present disclosure, the determined level of the one or more biomarkers is normalized to one or more physical and/or physiological characteristics of the newborn human patient, the standard or standards is normalized to the same one or more physical and/or physiological characteristics of normal newborn controls without congenital heart defect, and the normalized determined level of the one or more biomarkers is compared to the normalized standard or standards relating to the same one or more biomarkers, thereby detecting congenital heart defect in the newborn human patient, which determination is output to a user.

Computer-implemented methods of detection of CHD in a newborn human patient are provided according to aspects of the present disclosure which include: providing a determined level of: 1) sST2 and NT-proBNP; or 2) sST2 and Galectin-3; or 3) NT-proBNP and Galectin-3, or 4) all of sST2, NT-proBNP and Galectin-3, present in a dried blood sample of the newborn human patient; responsive to the determined level, assigning a score to the newborn human patient representative of detection of CHD by algorithmically comparing the determined level(s) to a standard or standards representing one or more normal newborn controls without congenital heart defect, thereby detecting congenital heart defect in the newborn human patient. According to aspects of the present disclosure, the determined level of the two or more biomarkers is normalized to one or more physical and/or physiological characteristics of the newborn human patient, the standard or standards is normalized to the same one or more physical and/or physiological characteristics of normal newborn controls without congenital heart defect, and the normalized determined level of the two or more biomarkers is compared to the normalized standard or standards relating to the same two or more biomarkers, thereby detecting congenital heart defect in the newborn human patient, which determination is output to a user.

### BRIEF DESCRIPTION OF THE DRAWINGS

FIG. 1A is a graph illustrating test performance of dried blood spot (DBS) analyses using the cardiovascular biomarkers NT-pro-BNP and sST2 in 82 healthy controls and 64 newborns with congenital heart defect (CHD) who passed pulse oximetry screening without signs of CHD; Receiver Operating Characteristics (ROC) analysis shows an area under the curve (AUC) of 0.92 [0.86-0.97], indicating excellent screening test performance to detect CHD;

FIG. 1B is a graph illustrating test performance of dried blood spot (DBS) analyses using the cardiovascular biomarkers NT-pro-BNP and sST2 in combination with patient specific demographic data to create an algorithmic model to assess patient-specific risk; analysis contains 81 healthy controls and 47 newborns with CHD who passed pulse oximetry screening without signs of CHD; ROC analysis shows an AUC of 0.96 [0.91-0.99], indicating outstanding screening test performance to detect CHD;

FIG. 2A is a graph illustrating level of NT-proBNP in DBS where the blood was obtained from infants at 2 days, 3 days, or 4 days after birth between normal control individuals (no CHD) vs newborns with CHD;

FIG. 2B is a graph illustrating level of sST2 in DBS where the blood was obtained from infants at 2 days, 3 days, or 4 days after birth between normal control individuals (no CHD) vs newborns with CHD;

FIG. 2C is a graph illustrating level of Galectin-3 in DBS where the blood was obtained from infants at 2 days, 3 days, or 4 days after birth between normal control individuals (no CHD) vs newborns with CHD;

FIG. 3A is a graph illustrating levels of NT-proBNP in DBS, where the blood was obtained from infants at 2 days, 3 days, or 4 days after birth, in normal control individuals (no CHD) (two lower lines) vs newborns with CHD (two upper lines) by gender (black=Male, grey=Female);

FIG. 3B is a graph illustrating levels of sST2 in DBS where the blood was obtained from infants at 2 days, 3 days, or 4 days after birth in normal control individuals (no CHD) (two lower lines) vs newborns with CHD (two upper lines) by gender (black=Male, grey=Female);

FIG. 3C is a graph illustrating levels of Galectin-3 in DBS where the blood was obtained from infants at 2 days, 3 days, or 4 days after birth in normal control individuals (no CHD) (two lower lines) vs newborns with CHD (two upper lines) by gender (black=Male, grey=Female);

FIG. 4 is a graph illustrating combinations of biomarkers, NT-proBNP, sST2, and Galectin-3, identify CHD cases in newborns which are missed by routine POX (pulse oximetry) screening;

FIG. 5A is a graph illustrating screening performance of NT-proBNP alone, without POX screening; ROC plot analysis with AUC=0.91 and CI 95% (0.87, 0.95); 112 CHD cases and 89 controls;

FIG. 5B is a graph illustrating screening performance of sST2 alone, without POX screening; ROC plot analysis with AUC=0.91 and CI 95% (0.87, 0.95); 112 CHD cases and 89 controls;

FIG. 5C is a graph illustrating screening performance of Galectin-3 alone, without POX screening; ROC plot analysis with AUC=0.69 and CI 95% (0.62, 0.76); 112 CHD cases and 89 controls;

FIG. 5D is a graph illustrating screening performance of NT-proBNP alone, with POX screening; ROC plot analysis with AUC=0.91 and CI 95% (0.84, 0.96); 52 CHD cases and 81 controls;

FIG. 5E is a graph illustrating screening performance of sST2 alone, with POX screening; ROC plot analysis with AUC=0.93 and CI 95% (0.87, 0.97); 52 CHD cases and 81 controls;

FIG. 5F is a graph illustrating screening performance of Galectin-3 alone, with POX screening; ROC plot analysis with AUC=0.7 and CI 95% (0.62, 0.8); 52 CHD cases and 81 controls;

FIG. 6A is a graph illustrating screening performance of NT-proBNP and Galectin-3 in combination without POX screening; ROC plot analysis with 112 CHD cases and 89 controls, AUC=0.92 and CI 95% (0.88, 0.96);

FIG. 6B is a graph illustrating screening performance of sST2 and Galectin-3 in combination without POX screening; ROC plot analysis with 112 CHD cases and 89 controls, AUC=0.92 and CI 95% (0.88, 0.95);

FIG. 6C is a graph illustrating screening performance of sST2 and NT-proBNP in combination without POX screening; ROC plot analysis with 112 CHD cases and 89 controls, AUC=0.96 and CI 95% (0.93, 0.98);

FIG. 6D is a graph illustrating screening performance of sST2 and NT-proBNP and Galectin-3 in combination without POX screening; ROC plot analysis with 112 CHD cases and 89 controls, AUC=0.96 and CI 95% (0.83, 0.98);

FIG. 7A is a graph illustrating screening performance of NT-proBNP and Galectin-3 in combination, and with POX screening (POX positive dropped out first); ROC plot analysis in 52 CHD cases and 81 controls; AUC=0.92 and CI 95% (0.87, 0.96);

FIG. 7B is a graph illustrating screening performance of sST2 and Galectin-3 in combination, and with POX screening (POX positive dropped out first); ROC plot analysis in 52 CHD cases and 81 controls; AUC=0.93 and CI 95% (0.88, 0.97);

FIG. 7C is a graph illustrating screening performance of sST2 and NT-proBNP in combination, and with POX screening (POX positive dropped out first)); ROC plot analysis in 52 CHD cases and 81 controls; AUC=0.95 and CI 95% (0.9, 0.99);

FIG. 7D is a graph illustrating screening performance of sST2 and NT-proBNP and Galectin-3 in combination, and with POX screening (POX positive dropped out first)); ROC plot analysis in 52 CHD cases and 81 controls; AUC=0.95 and CI 95% (0.9, 0.98);

FIG. 8A is a graph illustrating a Pearson correlation between blood plasma and DBS sST2 assay in controls (n=82); r=0.83, p<0.01;

FIG. 8B is a graph illustrating a Bland-Altman agreement plot after logarithmic conversion of test results to achieve parametric distribution for blood plasma and DBS sST2 assay in controls (n=82). Bias: 2.31, SD: 0.40; lower limits of agreement (LoA): 1.54 (CI1.39-1.69); upper LoA 3.09 (CI2.94-3.23);

FIG. 9A is a graph illustrating a ROC analysis for combined NT-proBNP and sST2 DBS assays to detect high-risk CHD in newborns (cases=188, controls=97; area under the curve (AUC)=0.95 (0.93-0.98), TPR=true positive rate, FPR=false positive rate;

FIG. 9B is a graph illustrating ROC analysis for combined NT-proBNP and sST2 DBS assays in subgroup of coarctation cases that were not recognized by pulse oximetry screening (n=70) compared to controls (n=86); area under curve (AUC)=0.97 (0.94-0.99) TPR=true positive rate, FPR=false positive rate;

FIG. 10 is a graph showing the effect of age (in days after birth) on sST2 levels in blood samples collected from normal (non-CHD) human newborn patients;

FIG. 11 is a schematic illustration of a method of computer-implemented methods of detection of CHD in a newborn human patient including input (10) of a result or results of an assay for one or more biomarkers of CHD selected from the group consisting of: soluble Suppression of Tumorigenicity 2 protein (sST2), and Galectin-3 in a dried blood sample obtained from a newborn human patient; comparing (20) the assay result or results to a first reference dataset such as standard levels of one or more of sSt2, NT-proBNP, and Galectin-3 found in a comparable blood sample of normal (non-CHD) newborn human patients; and outputting detection or non-detection (30) of CHD in the newborn human patient based on the result of the comparison of 10 and 20;

FIG. 12 is a schematic illustration of a method of computer-implemented methods of detection of CHD in a newborn human patient including input (10) of a result or results of an assay for one or more biomarkers of CHD selected from the group consisting of: soluble Suppression of Tumorigenicity 2 protein (sST2), and Galectin-3 in a dried blood sample obtained from a newborn human patient; comparing (20) the assay result or results to a first reference dataset such as standard levels of one or more of sSt2, NT-proBNP, and Galectin-3 found in a comparable blood sample of normal (non-CHD) newborn human patients; input (40) of pulse oximetry data from the newborn human patient; comparing (50) the pulse oximetry data from the newborn human patient to a pulse oximetry standard such as pulse oximetry data from normal (non-CHD) newborn human patients; and outputting detection or non-detection (30) of CHD in the newborn human patient based on the result of the comparison of 20 and 50; and

FIG. 13 is a schematic illustration of a method of computer-implemented methods of detection of CHD in a newborn human patient including input (60) of a result or results of an assay for one or more biomarkers of CHD selected from the group consisting of: soluble Suppression of Tumorigenicity 2 protein (sST2), and Galectin-3 in a dried blood sample obtained from a newborn human patient, wherein the input further includes one or more of: birth weight, gestational age, sex of newborn, and day of testing after birth, of the newborn human patient from whom the sample was obtained such that the assay result or results are normalized with respect to one or more of: birth weight, gestational age, sex of newborn, and day of testing after birth, of the newborn human patient from whom the sample was obtained thereby normalizing the assay result or results (60); comparing (70) the normalized assay result or results to a normalized standard which includes a level of one or more of sSt2, NT-proBNP, and Galectin-3, found in normal (non-CHD) newborn human patients and associated with one or more of: birth weight, gestational age, sex of newborn, and day of testing after birth in normal (non-CHD) newborn human patients; and outputting a result of detection or non-detection (30) of CHD in the newborn human patient based on the result of the comparison of 60 and 70.

### DETAILED DESCRIPTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; CRISPR/Cas: A Laboratory Manual, Doudna and Mali (eds), Cold Spring Harbor Laboratory Press, Cold Spring Harbor, NY, USA, 2016; D. L. Nelson and M. M. Cox, Lehninger Principles of Biochemistry, 4th Ed., W. H. Freeman & Company, 2004; J.-H. Fuhrhop et al. (Eds.), Organic Synthesis, Concepts and Methods, 3rd Ed., Wiley-VCH Cerlag GmbH & Co. KGaA, 2003; Herdewijn, P. (Ed.), Oligonucleotide Synthesis: Methods and Applications, Methods in Molecular Biology, Humana Press, 2004; D. J. Taxman (ed.), siRNA Design, Methods and Protocols, Humana Press, 2012; Harlow, E. and Lane, D., Antibodies: A Laboratory Manual, Cold Spring Harbor Laboratory Press, 1988; J. D. Pound (Ed.) Immunochemical Protocols, Methods in Molecular Biology, Humana Press, 2nd ed., 1998; Chu, E. and Devita, V. T., Eds., Physicians' Cancer Chemotherapy Drug Manual, Jones & Bartlett Publishers, 2021; J. M. Kirkwood et al., Eds., Current Cancer Therapeutics, 4th Ed., Current Medicine Group, 2001; A Adejare (Ed.), Remington: The Science and Practice of Pharmacy, Elsevier, 23rd Ed., 2021; L. V. Allen, Jr. et al., Ansel's Pharmaceutical Dosage Forms and Drug Delivery Systems, 11th Ed., Wolters Kluwer, 2016; and L. Brunton et al., Goodman & Gilman's The Pharmacological Basis of Therapeutics, McGraw-Hill Education, 13th Ed., 2018.

The singular terms “a,” “an,” and “the” are not intended to be limiting and include plural referents unless explicitly stated otherwise or the context clearly indicates otherwise.

The terms “includes,” “comprises,” “including,” “comprising,” “has,” “having,” and grammatical variations thereof, when used in this specification, are not intended to be limiting, and specify the presence of stated features, elements, and/or components, but do not preclude the presence or addition of one or more other features, elements, components, and/or groups thereof.

The term “about” as used herein in reference to a number is used herein to include numbers which are greater, or less than, a stated or implied value by 1%, 5%, 10%, or 20%.

Particular combinations of features are recited in the claims and/or disclosed in the specification, and these combinations of features are not intended to limit the disclosure of various aspects. Combinations of such features not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a alone; b alone; c alone, a and b, a, b, and c, b and c, a and c, as well as any combination with multiples of the same element, such as a and a; a, a, and a; a, a, and b; a, a, and c; a, b, and b; a, c, and c; and any other combination or ordering of a, b, and c).

The terms “first,” “second,” and the like are used herein to describe various features or elements, but these features or elements are not intended to be limited by these terms, but are only used to distinguish one feature or element from another feature or element. Thus, a first feature or element could be termed a second feature or element, and vice versa, without departing from the teachings of the present disclosure.

Methods of detection of CHD in a newborn human patient are provided according to aspects of the present disclosure which include performing an assay for one or more biomarkers of CHD, wherein the one or more biomarkers have a role in myocardial stretch and matrix remodeling.

The term “congenital heart defect” as used herein refers to critical congenital heart defects including, but not limited to: coarctation of the aorta, double-outlet right ventricle, d-transposition of the great arteries, Ebstein anomaly, hypoplastic left heart syndrome, interrupted aortic arch, pulmonary atresia (with intact septum), single ventricle, total anomalous pulmonary venous return, tetralogy of Fallot, tricuspid atresia, and truncus arteriosus. The term “congenital heart defect” is abbreviated as “CHD” herein.

Methods of detection of CHD in a newborn human patient are provided according to aspects of the present disclosure which include performing an assay for one or both biomarkers of CHD selected from the group consisting of: soluble Suppression of Tumorigenicity 2 protein (sST2), and Galectin-3, or 1) both sST2, and N-terminal pro b-type natriuretic peptide (NT-proBNP), or 2) both NT-proBNP and Galectin-3, or 3) all of sST2, NT-proBNP, and Galectin-3, in a dried blood sample obtained from the newborn human patient, thereby determining a level of the one or more biomarkers present in the dried blood sample.

Human ST2 (Suppression of Tumorigenicity 2) is a well-known member of the interleukin-1 receptor family and it is also known as interleukin-1 receptor-like 1 (IL1RL-1). ST2 exists in two isoforms: 1) a transmembrane or cellular form (ST2L) and 2) a soluble or circulating form known as “soluble Suppression of Tumorigenicity 2” (abbreviated sST2): KFSKQSWGLENEALIVRCPRQGKPSYTVDWYYSQTNKSIPTQERNRVFASGQLLKFLPA AVADSGIYTCIVRSPTFNRTGYANVTIYKKQSDCNVPDYLMYSTVSGSEKNSKIYCPTID LYNWTAPLEWFKNCQALQGSRYRAHKSFLVIDNVMTEDAGDYTCKFIHNENGANYSV TATRSFTVKDEQGFSLFPVIGAPAQNEIKEVEIGKNANLTCSACFGKGTQFLAAVLWQLN GTKITDFGEPRIQQEEGQNQSFSNGLACLDMVLRIADVKEEDLLLQYDCLALNLHGLRR HTVRLSRKNPIDHHS (SEQ ID NO:1)

Human Galectin-3 is well-known as a 249 amino acid protein which is a member of the galectin family of carbohydrate binding proteins: MADNFSLHDALSGSGNPNPQGWPGAWGNQPAGAGGYPGASYPGAYPGQAPPGAYPG QAPPGAYPGAPGAYPGAPAPGVYPGPPSGPGAYPSSGQPSATGAYPATGPYGAPAGPLI VPYNLPLPGGVVPRMLITILGTVKPNANRIALDFQRGNDVAFHFNPRFNENNRRVIVCNT KLDNNWGREERQSVFPFESGKPFKIQVLVEPDHFKVAVNDAHLLQYNHRVKKLNEISK LGISGDIDLTSASYTMI (SEQ ID NO:2)

Human N-terminal pro b-type natriuretic peptide (NT-proBNP) is well-known as a non-glycosylated polypeptide chain containing 76 amino acids and having a molecular mass of approximately 8.5 kDa: HPLGSPGSASDLETSGLQEQRNHLQGKLSELQVEQTSLEPLQESPRPTGVWKSREVATE GIRGHRKMVLYTLRAPR (SEQ ID NO:3)

The terms “biomarker” and “biomarkers” as used herein refers to one or more of human sST2, human NT-proBNP, and human Galectin-3 as represented in the amino acid sequences shown herein or a variant thereof wherein the variant has at least 90%, 91%, 92%, 93%, 94%, 95%, 96%, 97%, 98%, 99%, or greater, identity to the reference sequences shown herein.

To determine the percent identity of two amino acid sequences, the sequences are aligned for optimal comparison purposes (e.g., gaps can be introduced in the sequence of a first amino acid for optimal alignment with a second amino acid). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, PNAS 87:2264 2268, modified as in Karlin and Altschul, 1993, PNAS. 90:5873 5877. Such an algorithm is incorporated into the NBLAST and XBLAST programs of Altschul et al., 1990, J. Mol. Biol. 215:403. BLAST protein searches are performed with the XBLAST program parameters set, e.g., to score 50, wordlength=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389 3402. Alternatively, PSI BLAST is used to perform an iterated search which detects distant relationships between molecules (Id.). When utilizing BLAST, Gapped BLAST, and PSI Blast programs, the default parameters of the respective programs (e.g., of XBLAST and NBLAST) are used (see, e.g., the NCBI website). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

A variant may include one or more conservative amino acid substitutions.

Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

According to aspects, a method of detection of CHD in a newborn human patient includes comparing the determined level of biomarkers: sST2 alone, Galectin-3 alone, both sST2 and Galectin-3, both sST2 and NT-proBNP, both NT-proBNP and Galectin-3, or all of sST2, NT-proBNP, and Galectin-3, present in a dried blood sample obtained from the newborn human patient with a standard representing a normal newborn control without CHD, thereby determining whether the determined level is increased or decreased compared to the standard, wherein an increased level of the one or more biomarkers present in the dried blood sample, compared with a standard representing a normal newborn control without CHD, indicates detection of CHD in the newborn human patient.

The term “increased level” as used herein refers to a detectable increase compared to the standard. The increase may be an increase of about 1%, about 5%, about 10%, about 20%, about 30%, about 40%, about 50%, about 60%, about 70%, about 80%, about 90%, about 95%, about 99%, about 100%, or more, such as about 150%, i.e. 1.5-fold, about 2-fold, about 3-fold, about 4-fold, about 5-fold, about 6-fold, about 7-fold, about 8-fold, about 9-fold, about 10-fold, or more, compared to the standard.

Methods of detection of CHD in a newborn human patient are provided according to aspects of the present disclosure which include performing an assay for two or more biomarkers of CHD selected from the group consisting of: sST2, NT-proBNP, and Galectin-3 in a dried blood sample obtained from the newborn human patient.

Methods of detection of CHD in a newborn human patient are provided according to aspects of the present disclosure which include performing an assay for all of: sST2, NT-proBNP, and Galectin-3, in a dried blood sample obtained from the newborn human patient.

Methods of detection of CHD in a newborn human patient are provided according to aspects of the present disclosure which include performing an assay for both sST2 and NT-proBNP.

According to aspects of the present disclosure, the assay is a binding assay which includes specific binding of a binding agent to an analyte, wherein the analyte is sST2, NT-proBNP, or Galectin-3.

The term “binding agent” as used herein refers to an agent characterized by specific binding to a specified analyte. The phrase “specific binding” and grammatical equivalents as used herein in reference to binding of a binding agent to a specified analyte refers to binding of the binding agent to the specified analyte without substantial binding to other substances present in a sample to be analyzed.

The term “binding” refers to a physical or chemical interaction between a binding agent and the analyte. Binding includes, but is not limited to, ionic bonding, non-ionic bonding, covalent bonding, hydrogen bonding, hydrophobic interaction, hydrophilic interaction, and Van der Waals interaction.

Specific binding may refer to a binding agent that binds to sST2, NT-proBNP, or Galectin-3 with greater affinity, greater avidity, and/or greater duration, than to other substances. According to aspects of the present disclosure, a binding agent specifically binds to its analyte when it has an equilibrium dissociation constant, K_{D}, for its target in the range of about 10⁻⁴ to about 10⁻¹², i.e. a K_{D} of about 10⁻⁴, about 10⁻⁵, about 10⁻⁶, about 10⁻⁷, about 10⁻⁸, about 10⁻⁹, about 10⁻¹⁰, about 10⁻¹¹, or about 10⁻¹². Binding affinity of a binding agent can be determined by Scatchard analysis such as described in P. J. Munson and D. Rodbard, Anal. Biochem., 107:220-239, 1980 or by other methods such as Biomolecular Interaction Analysis using plasmon resonance.

Binding agents specific for sST2, NT-proBNP, or Galectin-3 may be obtained from commercial sources or generated for use in methods of the present disclosure according to well-known methodologies.

A binding agent according to aspects of the present disclosure may be an aptamer. The term “aptamer” refers to a nucleic acid or peptide that substantially specifically binds to a specified substance. In the case of a nucleic acid aptamer, the aptamer is characterized by binding interaction with a target other than Watson/Crick base pairing or triple helix binding with a second and/or third nucleic acid. Such binding interaction may include Van der Waals interaction, hydrophobic interaction, hydrogen bonding and/or electrostatic interactions, for example. Techniques for identification and generation of aptamers is known in the art as described, for example, in F. M, Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; and J. Sambrook and D. W. Russell, Molecular Cloning: A Laboratory Manual, Cold Spring Harbor Laboratory Press, 3rd Ed., 2001.

Methods of detection of a biomarker in a sample according to aspects of the present invention include contacting a sample containing or suspected of containing the biomarker under binding conditions with a biomarker-specific binding agent and a detectable label, wherein the detectable label is directly or indirectly attached to the biomarker-specific binding agent.

Methods of detection of a biomarker in a sample according to aspects of the present invention include contacting a sample containing or suspected of containing the biomarker under binding conditions with an a biomarker-specific antibody, or an antigen-binding fragment thereof, and a detectable label, wherein the detectable label is directly or indirectly attached to the biomarker-specific antibody, or an antigen-binding fragment thereof.

The term “detectable label” refers to any atom or moiety that can provide a detectable signal and which can be attached to a binding agent, such as a primary or secondary antibody or antigen binding fragment, or analyte. Examples of such detectable labels include fluorescent moieties, chemiluminescent moieties, bioluminescent moieties, ligands, particles, latex particles, luminescent particles, magnetic particles, fluorescent particles, colloidal gold, enzymes, enzyme substrates, radioisotopes, and chromophores. Where a particle is included in a detectable label, such particles can be of any shape, size, composition, or physiochemical characteristics compatible with assay conditions. The particles of a detectable label can be microparticles having a diameter of less than one millimeter, for example, a size ranging from about 0.1 to about 1,000 micrometers in diameter, inclusive, such as about 3-25 microns in diameter, inclusive, or about 5-10 microns in diameter, inclusive. The particles of a detectable label can be nanoparticles having a diameter from about 1 nanometer (nm) to about 100,000 nm in diameter, inclusive, for example, a size ranging from about 10-1,000 nm, inclusive, or for example, a size ranging from 200-500 nm, inclusive.

For a DELFIA format immunoassay, a detectable label can be europium.

For a chemiluminescence immunoassay, a detectable label can be an enzyme, such as horseradish peroxidase or alkaline phosphatase, which can act on a chemiluminescent substrate, such as luminol or adamantyl 1,2-dioxetane aryl phosphate (AMPPD), respectively.

For a chemiluminescence immunoassay, a detectable label can be a chemiluminescence reagent, such as acridinium ester, that emits light in the presence of a trigger without participation of an enzyme. Triggers for chemiluminescence where the label is a non-enzyme chemiluminescence reagent, such as an acridinium ester, include, but are not limited to, basic H₂O₂, e.g. a solution of H₂O₂ and NaOH or reductive triggering materials such as zinc metal and/or reduced forms of ferric and cupric salts.

Any appropriate method, including but not limited to, direct or indirect detection using a spectroscopic method, an optical method, a photochemical method, a biochemical method, an enzymatic method, an electrical method, am isotopic method, a magnetic method, an energetic method, and/or an immunochemical method, is used to detect a detectable label in an assay according to aspects of the present disclosure.

According to aspects of the present disclosure, the binding agent is an antibody, or an antigen-binding fragment of an antibody, and the assay is an immunoassay.

Immunoassays can be used to assay one or both of sST2, and Galectin-3 as biomarkers of CHD. Immunoassays can be used to assay 1) both sST2, and NT-proBNP), or 2) both NT-proBNP and Galectin-3, or 3) all of sST2, NT-proBNP, and Galectin-3 in a dried blood sample.

Immunoassays used include, but are not limited to, enzyme-linked immunosorbent assay (ELISA), enzyme-linked immunofiltration assay (ELIFA), flow cytometry, immunoblot, immunoprecipitation, immunohistochemistry, immunocytochemistry, luminescence immunoassay (LIA), chemiluminescence immunoassay (CLIA), fluorescence immunoassay (FIA), DELFIA® (dissociation-enhanced lanthanide fluorescence immunoassay), and radioimmunoassay. Details of immunoassays are described in published references, illustratively including Wild, D., The Immunoassay Handbook, 4th Ed., Elsevier Science, 2013; Vashist et al., Handbook of Immunoassay Technologies, Elsevier Science, 2018; Gosling, J. P., Immunoassays: A Practical Approach, Practical Approach Series, Oxford University Press, 2005; E. Harlow and D. Lane, Antibodies: A Laboratory Manual, Cold Spring Harbor Laboratory Press, 1988; F. Breitling and S. Dirbel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B. K. C. Lo, Antibody Engineering: Methods and Protocols, Methods in Molecular Biology, Humana Press, 2003; F. M. Ausubel et ah, Eds., Short Protocols in Molecular Biology, Current Protocols, Wiley, 2002; Ormerod, M. G., Flow Cytometry: a practical approach, Oxford University Press, 2000; and Givan, A. L., Flow Cytometry: first principles, Wiley, New York, 2001.

The term “antibody” is used herein in its broadest sense and includes single antibodies, single antigen-binding fragments, mixtures of antibodies, mixtures of antigen-binding fragments and mixtures of antigen-binding fragments and antibodies, characterized by specific binding to an antigen. An antibody included in methods according to aspects of the present disclosure may be a polyclonal antibody, a monoclonal antibody, a chimeric antibody, a humanized antibody, and/or an antigen-binding antibody fragment of any thereof. An antibody included in methods in particular aspects of the present disclosure includes a standard intact immunoglobulin having four polypeptide chains including two heavy chains (H) and two light chains (L) linked by disulfide bonds. An antibody included in methods in particular aspects of the present disclosure includes an antigen-binding antibody fragments illustratively include an Fab fragment, an Fab′ fragment, an F(ab′)2 fragment, an Fd fragment, an Fv fragment, an scFv fragment and a domain antibody (dAb), for example. In addition, the term antibody refers to antibodies of various classes including IgG, IgM, IgA, IgD and IgE, as well as subclasses, illustratively including for example human subclasses IgG1, IgG2, IgG3 and IgG4 and marine subclasses IgG1, IgG2, IgG2a, IgG2b, IgG3 and IgGM, for example.

In particular embodiments, an antibody which is characterized by specific binding to sST2, NT-proBNP, or Galectin-3 has a dissociation constant in the range of about 10⁻⁴ to about 10⁻¹², i.e. a K_{D} of about 10⁻⁴, about 10⁻⁵, about 10⁻⁶, about 10⁻⁷, about 10⁻⁸, about 10⁻⁹, about 10⁻¹⁰ about 10⁻¹¹, or about 10⁻¹². Binding affinity of an antibody can be determined by Scatchard analysis such as described in P. J. Munson and D. Rodbard, Anal. Biochem., 107:220-239, 1980 or by other methods such as Biomolecular Interaction Analysis using plasmon resonance. Antibodies may be tested for specific binding to the analyte by methods illustratively including ELISA, Western blot, and immunocytochemistry.

Antibodies specific for sST2, NT-proBNP, or Galectin-3 may be obtained from commercial sources or generated for use in methods of the present disclosure according to well-known methodologies.

Antibodies, antigen-binding fragments, and methods for their generation are known in the art, for instance, as described in Antibody Engineering, Kontemann, R. and Dubel, S. (Eds.), Springer, 2001; Harlow, E. and Lane, D., Antibodies: A Laboratory Manual, Cold Spring Harbor Laboratory Press, 1988; Ausubel. F. et al., (Eds.), Short Protocols in Molecular Biology, Wiley, 2002; J. D. Pound (Ed.) Immunochemical Protocols, Methods in Molecular Biology, Humana Press, 2nd ed., 1998; B. K. C. Lo (Ed.), Antibody Engineering: Methods and Protocols, Methods in Molecular Biology, Humana Press, 2003; and Kohler, G. and Milstein, C., Nature, 256:495-497 (1975).

According to aspects of the present disclosure, the immune assay is a competitive immunoassay in which a capture antibody specific for sST2, NT-proBNP, or Galectin-3 is coated on the internal surface of a reaction container which will contact a reaction mixture, such as in wells of a 96-well plate. In this format, about 100 ng to about 1500 ng is used to coat the inside of each well to be used. The amount of antibody used to coat a reaction container may be increased or decreased depending on variables such as the antibody used and/or the amount of sample to be assayed. Once the internal surface of the reaction container is coated, a DBS on a substrate can be introduced into the reaction container along with an elution buffer and a detection antibody which is specific for the same biomarker as the capture antibody. The detection antibody and the DBS/substrate are incubated in the elution buffer for an appropriate time, typically in the range of about 30 minutes to 4 hours, at a temperature in the range of about 10° C. to about 30° C., to elute the DBS material and allow the analyte to bind to the capture and detection antibodies. Following incubation, the substrate punch and liquid are removed from the well and the well is washed with wash buffer about 1 to 8 times followed by addition of an inducer solution (for DELFIA format) or substrate solution or trigger solution (for chemiluminescence or chromogen formats). Following incubation for about 1 to about 30 minutes, the assay readout is measured.

For a DELFIA format competitive immunoassay, the detection antibody is labeled with europium. For a chemiluminescence competitive immunoassay, the detection antibody is labeled with an enzyme, such as horseradish peroxidase or alkaline phosphatase, which can act on a chemiluminescent substrate, such as luminol or adamantyl 1,2-dioxetane aryl phosphate (AMPPD), respectively. For a chemiluminescence competitive immunoassay, the detection antibody is labeled with a chemiluminescence reagent, such as acridinium ester, that emits light in the presence of a trigger without participation of an enzyme. Triggers for chemiluminescence where the antibody is labeled with a non-enzyme chemiluminescence reagent, such as an acridinium ester include, but are not limited to, basic H₂O₂, e.g. a solution of H₂O₂ and NaOH or reductive triggering materials such as zinc metal and/or reduced forms of ferric and cupric salts.

For a chromogenic (or colorometric) competitive immunoassay, the detection antibody is labeled with enzyme, such as horseradish peroxidase, which can act on a chromogenic substrate for horseradish peroxidase, such as 3,3′,5,5′-tetramethylbenzidine (TMB) or 2,2′-azino-bis(3-ethylbenzothiazoline-6-sulfonic-acid) (ABTS) and o-phenylenediamine (oPD).

Methods of detection of CHD in a newborn human patient are provided according to aspects of the present disclosure which include performing an immunoassay for one or both of sST2, and Galectin-3 as biomarkers of CHD in a dried blood sample obtained from the newborn human patient putatively having CHD, thereby determining a level of the one or both biomarkers present in the dried blood sample. Methods of detection of CHD in a newborn human patient are provided according to aspects of the present disclosure which include performing an immunoassay for sST2 alone, Galectin-3 alone, both sST2 and Galectin-3, both sST2 and NT-proBNP, both NT-proBNP and Galectin-3, or all of sST2, NT-proBNP, and Galectin-3, in a dried blood sample obtained from the newborn human patient putatively having CHD, thereby determining a level of the specified biomarkers present in the dried blood sample.

Methods of detection of CHD in a newborn human patient are provided according to aspects of the present disclosure which include performing an immunoassay for both sST2 and NT-proBNP, together or separately, in a dried blood sample obtained from the newborn human patient putatively having CHD, thereby determining a level of the specified biomarkers present in the dried blood sample.

An assay result is compared to a control or standard according to preferred aspects of the invention.

The terms “control” and “standard” used interchangeably herein and are familiar to those of ordinary skill in the art and refer to any control or standard representing a normal newborn control without CHD that can be used for comparison to detect CHD. The control or standard may be determined prior to an assay of the present disclosure, in parallel, simultaneously, in a multiplex assay, or in another assay format. Standards may be used to obtain quantitative or semi-quantitative results indicative of the amount of one or more of sST2, NT-proBNP, and Galectin-3 in a newborn human patient DBS sample from a newborn human patient whose CHD status is unknown.

According to aspects of the present disclosure the standard representing a normal newborn control without CHD is, or includes, a reference level of one or more of sST2, NT-proBNP, and Galectin-3, previously determined in a control comparable non-CHD newborn DBS sample, determined in the same assay as the determination of sST2 alone, Galectin-3 alone, both sST2 and Galectin-3, both sST2 and NT-proBNP, both NT-proBNP and Galectin-3, or all of sST2, NT-proBNP, and Galectin-3, in the newborn human patient DBS sample from a newborn human patient whose CHD status is unknown, for example, by spiking the newborn human patient DBS sample from a newborn human patient whose CHD status is unknown with a known amount of labeled standard), or determined in parallel in a control comparable non-CHD newborn DBS sample.

The standard can be stored in a print or electronic medium for recall and comparison to a result of an assay of the present disclosure of a DBS sample from a newborn human patient whose CHD status is unknown.

According to aspects of the present disclosure, a method of detection of CHD in a newborn human patient includes comparing the determined level of the one or more biomarkers present in the dried blood sample with a standard representing a normal newborn control without CHD, thereby determining whether the determined level is increased or decreased compared to the standard, wherein an increased level of the one or more biomarkers present in the dried blood sample compared with a standard representing a normal newborn control without CHD indicates detection of CHD in the newborn human patient.

Methods of detection of CHD in a newborn human patient are provided according to aspects of the present disclosure which include performing an immunoassay for sST2 alone, Galectin-3 alone, both sST2 and Galectin-3, both sST2 and NT-proBNP, both NT-proBNP and Galectin-3, or all of sST2, NT-proBNP, and Galectin-3, in a dried blood sample obtained from the newborn human patient and comparing the determined level(s) of the one or more biomarkers present in the dried blood sample with a standard representing a normal newborn control without CHD, wherein an increased level of the one or more biomarkers present in the dried blood sample compared with a standard representing a normal newborn control without CHD indicates detection of CHD in the newborn human patient.

Methods of detection of CHD in a newborn human patient are provided according to aspects of the present disclosure which include performing an immunoassay for all of sST2, NT-proBNP, and Galectin-3 in a dried blood sample obtained from the newborn human patient and comparing the determined level(s) of the one or more biomarkers present in the dried blood sample with a standard representing a normal newborn control without CHD, wherein an increased level of all of sST2, NT-proBNP, and Galectin-3 present in the dried blood sample compared with a standard representing a normal newborn control without CHD indicates detection of CHD in the newborn human patient.

Methods of detection of CHD in a newborn human patient are provided according to aspects of the present disclosure which include performing an immunoassay for both sST2 and NT-proBNP, together or separately, in a dried blood sample obtained from the newborn human patient putatively having CHD, and comparing the determined level of sST2 and NT-proBNP present in the dried blood sample with a standard representing a normal newborn control without CHD, wherein an increased level of both sST2 and NT-proBNP present in the dried blood sample compared with the standard representing a normal newborn control without CHD indicates detection of CHD in the newborn human patient.

According to aspects of the present disclosure an included standard is one or more known amounts, in a clinically relevant range, of the analyte to be detected in the DBS, such as about 0, 1, 3, 10, 30, and 100 ng/mL and/or about 0, 0.1, 0.3, 1.0, 3.0, and 10 ng/ml of sST2, and/or NT-proBNP, and/or such as about 300, 500, 800, 900, and 1000 ng/ml Galectin-3.

According to aspects of the present disclosure, when sST2 is found to be greater than 5 ng/mL in a DBS from a newborn human patient, the level of sST2 is determined to be increased and the newborn human patient is thereby determined to have increased risk of CHD.

According to aspects of the present disclosure, when Galectin-3 is found to be greater than 800 ng/ml in a DBS from a newborn human patient, the level of Galectin-3 is determined to be increased and the newborn human patient is thereby determined to have increased risk of CHD.

According to aspects of the present disclosure, when sST2 is found to be greater than 5 ng/ml and NT-proBNP is found to be greater than 3 ng/ml in a DBS from a newborn human patient, the levels of sST2 and NT-proBNP are determined to be increased and the newborn human patient is thereby determined to have increased risk of CHD.

According to aspects of the present disclosure, when sST2 is found to be greater than 5 ng/ml and Galectin-3 is found to be greater than 800 ng/mL in a DBS from a newborn human patient, the levels of sST2 and Galectin-3 are determined to be increased and the newborn human patient is thereby determined to have increased risk of CHD.

According to aspects of the present disclosure, when NT-proBNP is found to be greater than 3 ng/ml and Galectin-3 is found to be greater than 800 ng/mL in a DBS from a newborn human patient, the levels of NT-proBNP and Galectin-3 are determined to be increased and the newborn human patient is thereby determined to have increased risk of CHD.

According to aspects of the present disclosure, when sST2 is found to be greater than 5 ng/mL NT-proBNP is found to be greater than 3 ng/mL, and Galectin-3 is found to be greater than 800 ng/mL in a DBS from a newborn human patient, the levels of sST2, NT-proBNP, and Galectin-3 are determined to be increased and the newborn human patient is thereby determined to have increased risk of CHD.

According to aspects of the present disclosure, a blood sample is obtained from the newborn human patient immediately after birth, or shortly after birth. The term “newborn human patient” as used herein refers to a human on the day of birth, one day after birth, 2, 3, 4, 5, 6, 7, 8, 9, 10, 11, 12, 13, 14, 15, 16, 17, 18, 19, 20, 21, 22, 23, 24, 25, 26, 27, or 28 days after birth. According to aspects of the present disclosure, a blood sample is obtained from the newborn human patient: on the day of birth, one day after birth, two days after birth, three days after birth, four days after birth, five days after birth, six days after birth, one week after birth, two weeks after birth, three weeks after birth, four weeks after birth, or later. According to aspects of the present disclosure, a blood sample is obtained from the newborn human patient: on the day of birth, one day after birth, 2, 3, 4, 5, 6, 7, 8, 9, 10, 11, 12, 13, 14, 15, 16, 17, 18, 19, 20, 21, 22, 23, 24, 25, 26, 27, or 28 days after birth.

The blood sample may be obtained from the newborn human patient from any source, including, but not limited to, capillary puncture such as by a heel prick or a hand prick, or any suitable skin puncture site; or venipuncture from any accessible vein.

The blood sample obtained from a newborn human patient has a volume in the range of about 1 microliter to about 10 milliliters depending on the age and weight of the newborn human patient, including about 1 microliter to about 2.5 milliliters from an 6 to 8 pound newborn human patient, about 1 microliter to about 3.5 milliliters from an 8 to 10 pound newborn human patient, about 1 microliter to about 5 milliliters from an 10 to 15 pound newborn human patient, and about 1 microliter to about 10 milliliters from an 16 to 41 pound newborn human patient. According to aspects of the present disclosure, the blood sample obtained from a newborn human patient has a volume in the range of about 1 microliter to about 1 milliliter for application to a substrate to produce a dried blood spot for analysis, such as about 1 microliter to about 500 microliters, about 2 microliters to about 250 microliters, about 3 microliters to about 200 microliters, about 5 microliters to about 100 microliters, about 5 microliters, about 10 microliters, about 15 microliters, about 20 microliters, about 25 microliters, about 30 microliters, about 40 microliters, about 45 microliters, about 50 microliters, about 55 microliters, about 60 microliters, about 65 microliters, about 70 microliters, about 75 microliters, about 80 microliters, about 85 microliters, about 90 microliters, about 95 microliters, or about 100 microliters.

A blood sample obtained from the newborn human patient, or a portion thereof, is applied to a substrate and dried.

A blood sample obtained from the newborn human patient, or a portion thereof, is applied to a substrate in one or more spots on the substrate and dried to produce DBS. Typically, each DBS contains about 5 microliters to about 10 microliters of the blood sample obtained from the newborn human patient.

The substrate may be any material compatible with a blood sample. Such substrates may include or consist of absorbent or non-absorbent materials, such as, but not limited to, cotton linter fiber filter paper, cellulose, cellulose acetate, cellulose ester, nitrocellulose, paper, silica, ceramic, nylon, polyamide, rayon, polyethersulfone, PTFE, glass fiber, polyester, polyethylene, polycarbonate, polyvinylidene difluoride, polypropylene, woven or non-woven natural or synthetic fibers, and combinations of any two or more of these. Substrates for drying a blood sample for immediate or later use in an assay according to aspects of the present disclosure may be obtained commercially. The substrate may be any shape or size convenient for application and drying of a blood sample, including planar, such as, but not limited to membranes; or three dimensional such as, but not limited to particles and fibers.

According to aspects of the present disclosure, the substrate is or includes one or more absorbent or non-absorbent materials, such as, but not limited to, cotton linter fiber filter paper, cellulose, cellulose acetate, cellulose ester, nitrocellulose, paper, silica, ceramic, nylon, polyamide, rayon, polyethersulfone, PTFE, glass fiber, polyester, polyethylene, polycarbonate, polyvinylidene difluoride, polypropylene, woven or non-woven natural or synthetic fibers, and combinations of any two or more of these.

According to aspects of the present disclosure, the substrate is a membrane which is or includes one or more absorbent or non-absorbent materials, such as, but not limited to, cotton linter fiber filter paper, cellulose, cellulose acetate, cellulose ester, nitrocellulose, paper, silica, ceramic, nylon, polyamide, rayon, polyethersulfone, PTFE, glass fiber, polyester, polyethylene, polycarbonate, polyvinylidene difluoride, polypropylene, woven or non-woven natural or synthetic fibers, and combinations of any two or more of these.

For use in an assay according to aspects of the present disclosure, the DBS is eluted from the substrate into an elution container. The container may be of any shape or size suitable for receiving an eluted DBS, such as a microtube or multiwell plate.

According to aspects of the present disclosure, the DBS, or a portion thereof, and the associated substrate is incubated in an aqueous elution medium in an elution container for a period of time sufficient to elute all, or a substantial portion of, the DBS. An aqueous elution medium is substantially inert with respect to the DBS and contact between the aqueous elution medium and the DBS on the substrate solubilizes the DBS, thereby removing all, or a substantial portion of, the DBS from the substrate. Non-limiting examples of an aqueous elution medium include aqueous physiological buffers such as, but not limited to, phosphate buffered saline, a Tris buffer, a Tricine buffer, a citrate buffer, a HEPES buffer, a carbonate buffer, a phosphate buffer, a MOPS buffer, a TAPS buffer, and an acetate buffer. Typically, the pH of the aqueous buffer is in the range of about pH 5.0 to about pH 9. According to aspects of the present disclosure, the pH of the aqueous buffer is about pH 5.0 to about pH 5.5, pH 6.0 to about pH 6.5, about pH 6.5 to about pH 7.0, about pH 7.0 to about pH 7.5, about pH 7.5 to about pH 8.0, about pH 8.0 to about pH 8.3, or about pH 8.3 to about pH 9.

As noted above, the DBS, or a portion thereof, and the associated substrate is incubated in an aqueous elution medium in an elution container for an elution time sufficient to elute all, or a substantial portion of, the DBS. The elution time is typically in the range of about 5 minutes to about 24 hours, such as about 5 minutes to about 2 hours, about 10 minutes to about 2 hours, about 15 minutes to about 2 hours, about 30 minutes to about 2 hours, about 45 minutes to about 2 hours, about 1 hour to about 2 hours, about 1 hour to about 4 hours, about 1 hour to about 6 hours, about 1 hour to about 12 hours, or about 1 hour to about 24 hours. The elution is performed at a temperature in the range of about 4° C. to about 37° C. According to aspects of the present disclosure, the elution is performed at a temperature in the range of about 15° C. to about 20° C., about 20° C. to about 25° C., or about 25° C. to about 30° C.

According to aspects of the present disclosure, a punch instrument is used to cut out a portion of the DBS and associated substrate. The punch instrument can be a manual punch instrument or automated punch instrument such as a Panthera Puncher (Revvity) which can automatically munch multiple DBS samples directly into a multiwell plate. Punch head sizes can be selected depending on the size of the container into which the punch will be placed, the amount of DBS on the substrate, and the amount of the DBS to be used in the assay. Example punch head sizes have a diameter of about 1.5 mm, 3.2 mm, 3.8 mm, 4.75 mm or 6.0 mm, and may be larger or smaller as desired. A punch taken from a typical DBS contains about 1 microliter, 2 microliters, 3 microliters, 4 microliters, 5 microliters, 6 microliters, 7 microliters, 8 microliters, 9 microliters, or 10 microliters, of the blood sample obtained from the newborn human patient which was dried onto the substrate, although more, or less, may be used.

According to aspects of the present disclosure, methods of detection of CHD in a newborn human patient are provided which further include assessment of the newborn human patient using pulse oximetry (POX).

The term “pulse oximetry” refers to a noninvasive measurement of oxygen saturation of the blood. A typical method of pulse oximetry is implemented using a pulse oximeter including a sensor that measures light absorption characteristics of hemoglobin, based on spectroscopic differences between oxygenated hemoglobin and deoxygenated hemoglobin, through the skin at an accessible location such as a finger, toe, or earlobe.

According to aspects of the present disclosure, methods of detection of CHD in a newborn human patient are provided which further include assessment of one or more physical characteristics of the newborn human patient. Examples of physical characteristics of the newborn human patient which may be assessed include but are not limited to, gestational age (GA) week of delivery, day of testing after birth, birthweight, and gender.

According to aspects of the present disclosure, methods of detection of CHD in a newborn human patient are provided which further include assessment of one or more physiological characteristics of the newborn human patient. Examples of physiological characteristics of the newborn human patient which may be assessed include but are not limited to, breathing effort, heart rate, muscle tone, reflexes, skin color (such as integrated in an Apgar score), blood pH, and jaundice.

According to aspects of the present disclosure, methods of detection of CHD in a newborn human patient are provided which further include normalization of assayed levels of sST2 alone, Galectin-3 alone, both sST2 and Galectin-3, both sST2 and NT-proBNP, both NT-proBNP and Galectin-3, or all of sST2, NT-proBNP, and Galectin-3, in a dried blood sample obtained from the newborn human patient, with one or more physical characteristics of the newborn human patient and/or one or more physiological characteristics of the newborn human patient.

According to aspects of the present disclosure, methods of detection of CHD in a newborn human patient are provided which further include normalization of assayed levels of: sST2 alone, Galectin-3 alone, both sST2 and Galectin-3, both sST2 and NT-proBNP, both NT-proBNP and Galectin-3, or all of sST2, NT-proBNP, and Galectin-3, in a dried blood sample obtained from the newborn human patient with one or more of: birth weight, gestational age, sex of newborn, and day of testing after birth of the newborn human patient.

The term “normalization” as used herein refers to adjustment of an assay result value, e.g. an assayed level of: sST2 alone, Galectin-3 alone, both sST2 and Galectin-3, both sST2 and NT-proBNP, both NT-proBNP and Galectin-3, or all of sST2, NT-proBNP, and Galectin-3, in a dried blood sample obtained from the newborn human patient, to account for a variation due to a variable other than presence or absence of CHD. According to aspects of the present disclosure, an assay result value is normalized with respect to one or more of: birth weight, gestational age, sex of newborn, and day of testing after birth of the newborn human patient, all of which reproducibly affect the level of sST2, NT-proBNP, and Galectin-3, in a dried blood sample of a newborn human patient.

According to aspects of the present disclosure, methods of detection of CHD in a newborn human patient are provided which further include normalization of assayed levels of: sST2 in a dried blood sample obtained from the newborn human patient with one or more of: birth weight, gestational age, sex of newborn, and day of testing after birth of the newborn human patient.

According to aspects of the present disclosure, methods of detection of CHD in a newborn human patient are provided which further include normalization of assayed levels of: Galectin-3 in a dried blood sample obtained from the newborn human patient with one or more of: birth weight, gestational age, sex of newborn, and day of testing after birth of the newborn human patient.

According to aspects of the present disclosure, methods of detection of CHD in a newborn human patient are provided which further include normalization of assayed levels of: both sST2 and NT-proBNP in a dried blood sample obtained from the newborn human patient with one or more of: birth weight, gestational age, sex of newborn, and day of testing after birth of the newborn human patient.

According to aspects of the present disclosure, methods of detection of CHD in a newborn human patient are provided which further include normalization of assayed levels of: both sST2 and Galectin-3 in a dried blood sample obtained from the newborn human patient with one or more of: birth weight, gestational age, sex of newborn, and day of testing after birth of the newborn human patient.

According to aspects of the present disclosure, methods of detection of CHD in a newborn human patient are provided which further include normalization of assayed levels of: both NT-proBNP and Galectin-3 in a dried blood sample obtained from the newborn human patient with one or more of: birth weight, gestational age, sex of newborn, and day of testing after birth of the newborn human patient.

According to aspects of the present disclosure, methods of detection of CHD in a newborn human patient are provided which further include normalization of assayed levels of: sST2, NT-proBNP, and Galectin-3 in a dried blood sample obtained from the newborn human patient with one or more of: birth weight, gestational age, sex of newborn, and day of testing after birth of the newborn human patient.

Assays according to aspects of the present disclosure may be performed as singleplex assays, i.e. as an assay for one biomarker. The results of multiple singleplex assays may then be considered together to detect CHD in the newborn human patient.

Optionally, assays according to aspects of the present disclosure may be performed as multiplex assays, i.e. as a combined assay for two or more of the biomarkers together: both sST2 and Galectin-3, both sST2 and NT-proBNP, both NT-proBNP and Galectin-3, or all of sST2, NT-proBNP, and Galectin-3.

Once a CHD is detected using an assay of the present disclosure, the newborn human patient treated to further characterize, and/or ameliorate the CHD. According to aspects of the present disclosure, an additional test is performed to further characterize the CHD, such as one or more of: an electrocardiogram, a chest x-ray, an echocardiogram, a transesophageal echocardiogram, cardiac catheterization, an angiogram, magnetic resonance imaging of the heart, and computerized tomography of the heart.

One or more treatments is performed to correct and/or ameliorate the CHD according to aspects of the present disclosure, such as administration of medication and/or surgery.

The terms “treating” and “treatment” used to refer to treatment of a CHD in a newborn human patient include: inhibiting or ameliorating the CHD in the subject, such as reducing or ameliorating a sign or symptom of the CHD in the subject, or eliminating a sign or symptom of the CHD in the subject.

Methods of treatment of a CHD are provided according to aspects of the present disclosure which include performing an assay for sST2 alone, Galectin-3 alone, both sST2 and Galectin-3, both sST2 and NT-proBNP, both NT-proBNP and Galectin-3, or all of sST2, NT-proBNP, and Galectin-3, in a dried blood sample obtained from the newborn human patient and comparing the determined level(s) of the one or more biomarkers present in the dried blood sample with a standard representing a normal newborn control without CHD, wherein an increased level of the one or more biomarkers present in the dried blood sample compared with a standard representing a normal newborn control without CHD indicates detection of CHD in the newborn human patient; and treating a detected CHD in the patient.

Methods of treatment of a CHD are provided according to aspects of the present disclosure which further include performing an assay to detect CHD following treatment of the detected CHD to confirm successful treatment by detection of a decrease in level of sST2 alone, Galectin-3 alone, both sST2 and Galectin-3, both sST2 and NT-proBNP, both NT-proBNP and Galectin-3, or all of sST2, NT-proBNP, and Galectin-3. The assay to detect CHD following treatment may be performed one or more times at various intervals following treatment, such as 1 day to 1 year following treatment, including 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, or 12 months following treatment.

Particular treatments include surgical repair of coarctation of the aorta, surgical ligation of patent ductus arteriosus, surgical repair of atrial septal defect, surgical repair of ventricular septal defect, surgical repair of tetralogy of Fallot, surgical repair of transposition of the great vessels, surgical repair of truncus arteriosus, surgical repair of tricuspid atresia, surgical repair of total anomalous pulmonary venous return, and surgical repair of hypoplastic left heart.

Cardiac catheterization is used to repair atrial septal defect and patent ductus arteriosus.

Indomethicin and/or ibuprofen can be used to treat patent ductus arteriosus.

Detailed surgical procedures are described in Bernstein D. General principles of treatment of congenital heart disease, Kliegman R M et al., eds. Nelson Textbook of Pediatrics. 21st ed. Philadelphia, PA: Elsevier; 2020, chapter 461; Stout K K, Daniels C J, Aboulhosn J A, et al. 2018 AHA/ACC guideline for the management of adults with congenital heart disease: A report of the American College of Cardiology/American Heart Association task force on clinical practice guidelines. J Am Coll Cardiol. 2019;73(12):e81-e192; and Well A, and Fraser C D. Congenital heart disease. In: Townsend C M et al., eds. Sabiston Textbook of Surgery. 21st ed. St Louis, MO: Elsevier; 2022, chapter 59.

Kits for detection of CHD in a newborn human patient are provided according to aspects of the present disclosure which include one or more reagents for performing an assay for one or more biomarkers of CHD selected from the group consisting of: sST2 alone, Galectin-3 alone, both sST2 and Galectin-3, both sST2 and NT-proBNP, both NT-proBNP and Galectin-3, or all of sST2, NT-proBNP, and Galectin-3, in a dried blood sample obtained from the newborn human patient, to determine a level of the one or more biomarkers present in the dried blood sample.

A kit for detection of CHD in a newborn human patient according to aspects of the present disclosure includes one or more reagents for performing an assay for sST2 alone, Galectin-3 alone, both sST2 and Galectin-3, both sST2 and NT-proBNP, both NT-proBNP and Galectin-3, or all of sST2, NT-proBNP, and Galectin-3, in a dried blood sample obtained from the newborn human patient, to determine a level of the sST2 alone, Galectin-3 alone, both sST2 and Galectin-3, both sST2 and NT-proBNP, both NT-proBNP and Galectin-3, or all of sST2, NT-proBNP, and Galectin-3, present in the dried blood sample, wherein an increased level of sST2 alone, Galectin-3 alone, both sST2 and Galectin-3, both sST2 and NT-proBNP, both NT-proBNP and Galectin-3, or all of sST2, NT-proBNP, and Galectin-3, in the a dried blood sample obtained from the newborn human patient compared to a standard is indicative of a CHD.

A kit according to aspects of the present disclosure includes one or more antibodies specific for sST2, one or more antibodies specific for Galectin-3, and one or more antibodies specific for NT-proBNP. A kit according to aspects of the present disclosure includes one or more antibodies specific for sST2 and one or more antibodies specific for NT-proBNP; or one or more antibodies specific for sST2 and one or more antibodies specific for Galectin-3 or one or more antibodies specific for NT-proBNP and one or more antibodies specific for Galectin-3. A kit according to aspects of the present disclosure includes one or more antibodies specific for sST2 or one or more antibodies specific for NT-proBNP.

According to aspects of the present disclosure, a kit for detection of CHD in a newborn human patient includes one or more reagents or devices for collection and drying of a blood sample from a newborn human patient. A kit according to aspects of the present disclosure includes one or more reagents or devices for one or more of: collection, drying, transport, and storage, of a blood sample from a newborn human patient, such as a substrate for deposition of a blood sample to produce a DBS and/or a container therefore.

A kit according to aspects of the present disclosure includes one or more auxiliary components, such as one or more of: a standard, a secondary binding agent, such as a secondary antibody, one or more containers, an aqueous elution medium, an assay buffer, a diluent, a reconstituting agent, a wash solution, an enhancer or inducer, a blocking solution (e.g. for blocking non-specific binding to an assay container) or a protein to be solubilized to prepare a blocking solution, and a detection system.

A standard included in a kit according to aspects of the present disclosure is one or more known amounts, in a clinically relevant range, of the analyte to be detected in a DBS, such as about 0, 1, 3, 10, 30, and 100 ng/mL and/or about 0, 0.1, 0.3, 1.0, 3.0, and 10 ng/ml of sST2, and/or NT-proBNP, and/or such as about 300, 500, 800, 900, and 1000 ng/ml Galectin-3. A standard included in a kit according to aspects of the present disclosure is included as a solution or as a ready to reconstitute solid.

A container included in a kit according to aspects of the present disclosure can be a container of any suitable shape, size, and composition compatible with a sample and/or reagents to be contained therein, such as inert with respect to a sample and/or reagents to be contained therein. Non-limiting examples of such containers include a microtube and a multiwell plate.

Liquids included in a kit according to aspects of the present disclosure, such as an aqueous elution medium, an assay buffer, a diluent, a reconstituting agent, and a wash solution may be, or include, water, or aqueous physiological buffers such as, but not limited to, phosphate buffered saline, a Tris buffer, a Tricine buffer, a citrate buffer, a HEPES buffer, a carbonate buffer, a phosphate buffer, a MOPS buffer, a TAPS buffer, and an acetate buffer. Typically, the pH of the aqueous buffer is in the range of about pH 5.0 to about pH 9. According to aspects of the present disclosure, the pH of the aqueous buffer is about pH 5.0 to about pH 5.5, pH 6.0 to about pH 6.5, about pH 6.5 to about pH 7.0, about pH 7.0 to about pH 7.5, about pH 7.5 to about pH 8.0, about pH 8.0 to about pH 8.3, or about pH 8.3 to about pH 9.

A blocking solution contains a blocking substance to block non-specific binding of the sample and/or an antibody to a reaction container. The blocking substance may include a protein, a non-ionic surfactant, or other additive which does not interfere with the desired assay. A blocking solution is illustratively serum, or a solution of serum albumin, such as bovine serum albumin, in an amount of about 1% to about 10% weight/volume (w/v). Optionally, a blocking protein, such as serum albumin, is provided as a solid for reconstitution in a buffer to prepare the blocking solution.

A detection system included in a kit according to aspects of the present disclosure includes one or more reagents for generating a detectable signal indicative of specific binding of a binding agent, such as: a chemiluminescent system including, an enzyme-labelled primary or secondary antibody and a substrate for the enzyme, wherein action of the enzyme on the substrate produces luminescence, such as alkaline phosphatase and adamantyl 1,2-dioxetane aryl phosphate (AMPPD) substrate, or horseradish peroxidase and luminol or a luminol derivative. An enhancer of luminescence signal, including: a phenol or a p-phenolic derivative, such as p-iodophenol, p-phenylphenol, 4-tert-butylphenol, 1-methyl-4-hydroxybenzene, 4-(4′-iodo)polyphenol, 4-(1,2,4-triazole-1-yl)phenol, 4-(1-imidazolyl)-pheol, 4-hydroxybi-phenyl), 4-hydroxy-4′iodopheyl, N-alkylated phenothiazine, ferricyanide/ferrocyanide, metallic ions, or a combination of two or more thereof, is included in a kit according to aspects of the present disclosure.

A detection system included in a kit according to aspects of the present disclosure includes one or more reagents for generating a detectable signal indicative of specific binding of a binding agent, such as: a DELFIA system including, for example, a low-pH inducer solution, and/or a primary or secondary detection antibody labelled with europium (Eu). An example of an included induced solution includes Triton X-1001, hydrochloric acid, and one or more chelators.

A detection system included in a kit according to aspects of the present disclosure includes one or more reagents for generating a detectable signal indicative of specific binding of a binding agent, such as: a colorimetric ELISA system including, for example, a colorimetric substrate for horseradish peroxidase, such as 3,3′,5,5′-tetramethylbenzidine (TMB) or 2,2′-azino-bis(3-ethylbenzothiazoline-6-sulfonic-acid) (ABTS) and o-phenylenediamine (oPD).

For a sandwich format assay kit, a container, such as a multiwell plate, may be included in a kit according to aspects of the present disclosure which is coated with an analyte-specific capture antibody. For singleplex assays, a container, such as a multiwell plate, is included in a kit according to aspects of the present disclosure wherein one or more wells is coated with an anti-sST2 capture antibody, an anti-Galectin-3 capture antibody, or an anti-NT-proBNP capture antibody. For multiplex assays, a container, such as a multiwell plate, is included in a kit according to aspects of the present disclosure wherein one or more wells is coated with both an anti-sST2 capture antibody and an anti-Galectin-3 antibody, both an anti-sST2 capture antibody and an anti-NT-proBNP capture antibody, both an anti-NT-proBNP capture antibody and an anti-Galectin-3 capture antibody or all of an anti-sST2 capture antibody, an anti-NT-proBNP capture antibody, and an anti-Galectin-3 capture antibody.

The amount of the anti-analyte capture antibody used to coat a container, such as a well of a multiwell plate, depends on the size of the container, such as the size of the well. Typically, for a standard 96-well microtiter plate having a maximum volume per well of 300 microliters, about 50 ng to about 1500 ng of an anti-analyte capture antibody is used to coat each well. The amount used can be scaled accordingly if a container with differently sized wells is used.

A corresponding detection antibody used in a sandwich format assay is typically in the range of about 50 ng/well to about 150 ng/well of a standard 96-well microtiter plate. The amount used can be scaled accordingly if a container with differently sized wells is used.

Computer-implemented methods of detection of CHD in a newborn human patient are provided according to aspects of the present disclosure which include providing a determined level of one or both biomarkers of CHD selected from the group consisting of: sST2 alone, Galectin-3 alone, or both sST2 and Galectin-3, both sST2 and NT-proBNP), or both NT-proBNP and Galectin-3, or all of sST2, NT-proBNP, and Galectin-3, present in a dried blood sample of the newborn human patient; responsive to the determined level, assigning a score to the newborn human patient representative of detection of CHD by algorithmically comparing the determined level to a standard representing a normal newborn control without CHD, thereby detecting CHD in the newborn human patient. According to aspects of the present disclosure, the score is output to a user via one or more output devices.

Computer-implemented methods of detection of CHD in a newborn human patient may be implemented as software, or a combination of hardware and software on one or more computer systems. According to aspects of the present disclosure, a step of comparing the determined level of the one or more biomarkers present in the dried blood sample with a standard in a method of detection of congenital heart defect (CHD) in a newborn human patient is performed with one or more computer processors of a computer system.

A computer system typically includes a processor, operating system, memory, bus, an interface, and storage. A computer system may be, for example, a personal computer, a workstation, a server, or a mobile computing device such as a cellular telephone.

A computer system according to aspects of the present disclosure may have a functionality distributed across multiple processing elements wherein the processing element refers to a process running on one piece of hardware or across multiple pieces of hardware.

An included processor performs a series of instructions that manipulate data. According to aspects of the present disclosure, the processor includes one or more processing elements, such as an ATHLON processor such as Zen-based Athlon processor or Ryzen processor (AMD Corp., Sunnyvale. Calif.), CORE i3, i5, i7, or i9 class microprocessor (Intel Corp., Santa Clara, Calif.), or one or more other general purpose processors could be included.

An included processor may include one or more microprocessors or controllers. The processor is connected to one or more other computer system components, such as a memory, by the bus.

An included memory operates to store programs and/or data. Examples of computer memory include any device for storing programs and/or data such as DRMA, SRAM, a disk drive, flash memory, or phase-change memory.

An included interface allows a user or other computer system to interact and exchange information with the computer system and may include an input device, an output device, or combination thereof. Examples of interfaces include a keyboard, mouse, microphone, touchscreen, display screen, speaker, printer, and the like.

According to aspects of the present disclosure, one or more input devices may receive input from one or more users and/or one or more software applications. According to aspects of the present disclosure, the input includes a test dataset, such as one or more results of an assay for detection of CHD in a newborn human patient including a level of sST2 alone, Galectin-3 alone, both sST2 and Galectin-3, both sST2 and NT-proBNP, both NT-proBNP and Galectin-3, or all of SST2, NT-proBNP, and Galectin-3, in a dried blood sample obtained from the newborn human patient. As mentioned above, Computer-implemented methods of detection of CHD in a newborn human patient are provided according to aspects of the present disclosure which include providing a determined level of one or both biomarkers of CHD selected from the group consisting of: sST2 alone, Galectin-3 alone, or both sST2 and Galectin-3, both sST2 and NT-proBNP), or both NT-proBNP and Galectin-3, or all of sST2, NT-proBNP, and Galectin-3, present in a dried blood sample of the newborn human patient, i.e. the test dataset; responsive to input of the test dataset, i.e. the determined level, assigning a score to the newborn human patient representative of detection of CHD by algorithmically comparing the determined level to a standard representing a normal newborn control without CHD, thereby detecting CHD in the newborn human patient.

According to aspects of the present disclosure, one or more output devices, such as a display screen, present a result of a method of detection of CHD in a DBS of a newborn human patient according to aspects of the present disclosure.

According to aspects of the present disclosure, one or more output devices, such as a display screen, present a graphical output representing one or more results of a method of detection of CHD in a DBS of a newborn human patient according to aspects of the present disclosure.

An included storage system may include a computer-readable/writable nonvolatile storage medium including instructions relating to a program to be executed by the processor.

According to aspects of the present disclosure the memory includes a first reference dataset, i.e. a standard or set of standards, wherein the first reference dataset includes standard levels of one or more of sSt2, NT-proBNP, and Galectin-3, found in normal (non-CHD) newborn human patients.

According to aspects of the present disclosure the memory further includes a second reference dataset, i.e. a normalized standard, which include standard levels of one or more of sSt2, NT-proBNP, and Galectin-3, found in normal (non-CHD) newborn human patients and associated with one or more of: birth weight, gestational age, sex of newborn, and day of testing after birth in normal (non-CHD) newborn human patients. The test dataset can correspondingly include one or more of: birth weight, gestational age, sex of newborn, and day of testing after birth, of the newborn human patient from whom the sample was obtained and the test dataset for that patient is normalized in accordance with the second reference dataset, producing a first normalized test result.

According to aspects of the present disclosure the memory further includes a third reference dataset, i.e. a normalized standard, which include standard levels of one or more of sSt2, NT-proBNP, and Galectin-3, found in normal (non-CHD) newborn human patients and associated with one or more of: breathing effort, heart rate, muscle tone, reflexes, skin color (such as integrated in an Apgar score), blood pH, and jaundice in normal (non-CHD) newborn human patients. The test dataset can include values for one or more of: breathing effort, heart rate, muscle tone, reflexes, skin color (such as integrated in an Apgar score), blood pH, and jaundice, of the newborn human patient from whom the sample was obtained and the test dataset for that patient is normalized in accordance with the third reference dataset, producing a second normalized test result.

The first and second normalized test results may be produced independently and it is not necessary to produce both the first and second normalized test results in order to detect CHD.

The normalized test result or results are compared with a normalized standard.

According to aspects of the present disclosure the memory further includes one or more additional reference datasets.

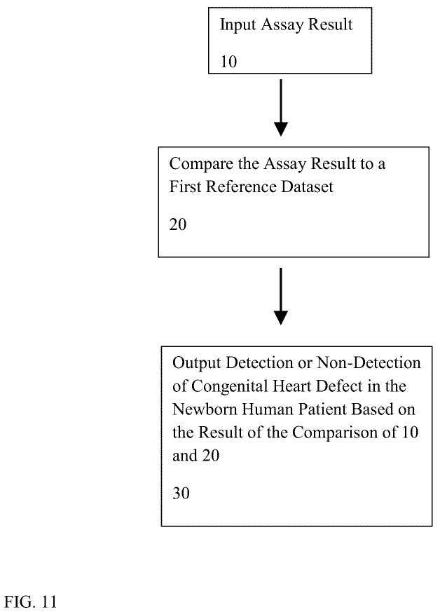

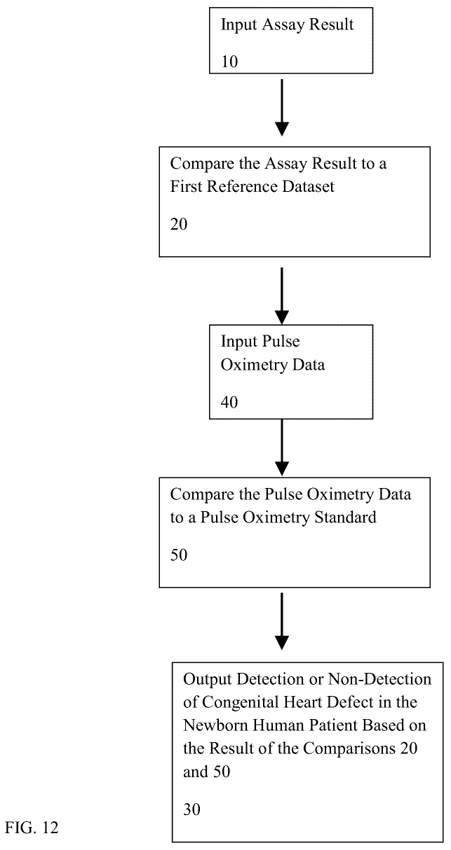

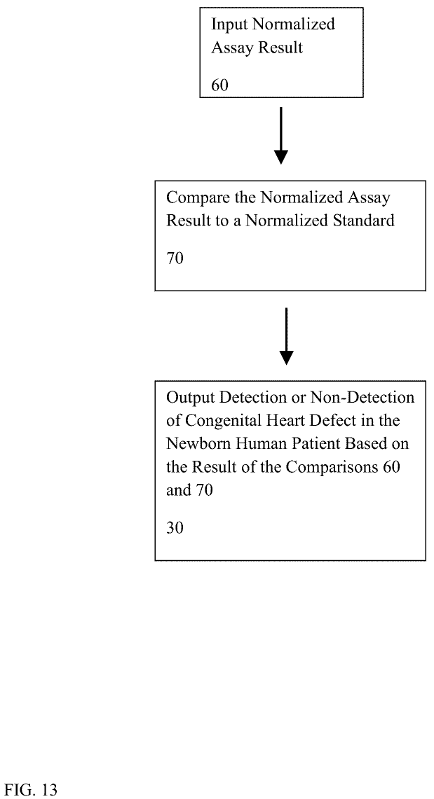

FIG. 11-13 schematically show computer-implemented methods of detection of CHD in a newborn human patient according to aspects of the present disclosure.

Embodiments of inventive compositions and methods are illustrated in the following examples. These examples are provided for illustrative purposes and are not considered limitations on the scope of inventive compositions and methods.

### EXAMPLES

### Example 1

Prospectively enrolled controls were compared to retrospectively identified CHD cases from pediatric cardiac surgical centers in Sweden. Controls were followed to ensure no late presentation of critical CHD occurred during infancy. Cases were verified using surgical records and perinatal data, including pulse oximetry screening (POX). Blinded DBS batch analysis obtained during day 2-7 of life was performed for amino-terminal pro-hormone of brain natriuretic peptide (NT-pro-BNP) and soluble Suppression of Tumorigenicity 2 protein (sST2). Receiver operating characteristics (ROC) were used to analyze results.

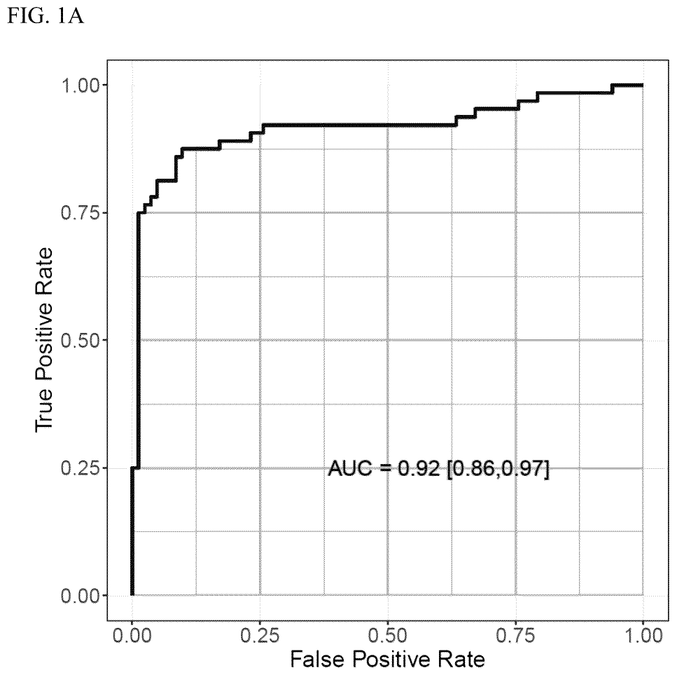

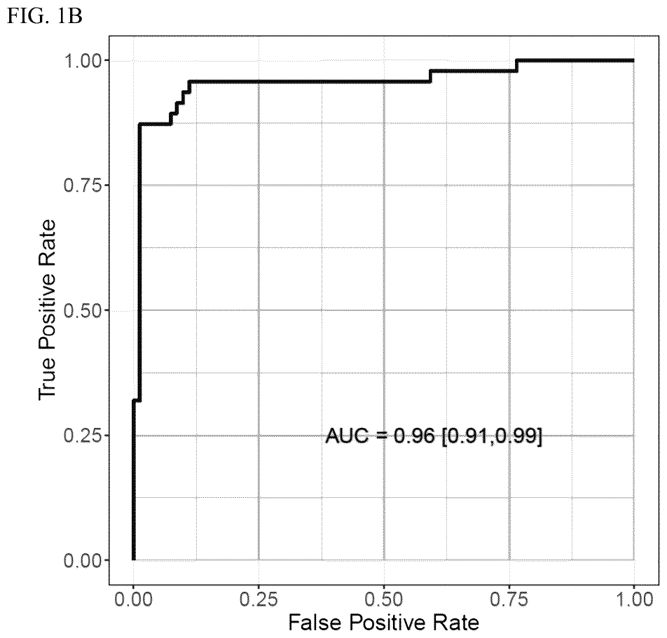

Results: 82 controls and 64 cases born at 36-42 weeks gestation, who passed POX without signs of CHD were analyzed. NT-pro-BNP and sST2 could be analyzed using <10 microliters of DBS samples collected during day 2-7 of life. Outstanding test performance was seen on ROC analyses to detect CHD in this cohort, AUC 0.92 [0.86-0.97] as shown in FIG. 1A.

Conclusion: Quantification of the biomarkers NT-pro-BNP and sST2 is feasible using DBS samples. Tests performed outstandingly to differentiate CHD from healthy controls, even without signs of CHD at time of POX.

### Example 2

Prospectively enrolled controls were compared to retrospectively identified CHD cases from pediatric cardiac surgical centers in Sweden. Controls were followed to ensure no late presentation of critical CHD occurred during infancy. Cases were verified using surgical records and perinatal data, including pulse oximetry screening (POX). Blinded DBS batch analysis obtained during day 2-7 of life was performed for amino-terminal pro-hormone of brain natriuretic peptide (NT-pro-BNP) and soluble Suppression of Tumorigenicity 2 protein (sST2). Receiver operating characteristics (ROC) were used to analyze results.

Results: Performance of dried blood spot (DBS) analyses using the cardiovascular biomarkers NT-pro-BNP and sST2 in combination with patient specific demographic data to create an algorithmic model to assess patient-specific risk were tested; analysis contains 81 controls and 47 cases born at 36-42 weeks gestation, who passed POX without signs of CHD were analyzed. NT-pro-BNP and sST2 could be analyzed using <10 microliters of DBS samples collected during day 2-7 of life.

Conclusion: Outstanding test performance was seen on ROC analysis shows an AUC of 0.96 [0.91-0.99], indicating outstanding screening test performance to detect CHD; as shown in FIG. 1B.

### Example 3

Samples: In this example, dried blood spots (DBS) were used that were collected from heal prick(s) onto a collection card. The DBS were assayed within 1 week (1-7 days) after application onto the DBS collection card.

For each well of a standard 96-well plate, a single standard sized, 3.2 mm punch, taken with DBS puncher instrument from a DBS spot dried on a DBS card for analysis.

Assays in this example were performed using an automated Genetic Screening Processor (GSP) instrument which allows for both DELFIA immunoassay and enzymatic assays:

A 96-well plate was coated with 500 ng/well of an analyte specific capture antibody. The capture antibodies used in this example were: NT-proBNP monoclonal antibody clone 15C4 from Hytest, sST2=Monoclonal antibody Clone 10201 SPTN-5 from Medix Biochemica, Galectin-3=Monoclonal antibody clone 10301 SPTN-5, from Medix Biochemica.

Analysis of NT-proBNP was performed in this example using a 1-step assay as follows:
Take an anti-NT-proBNP capture antibody-coated 96-well plate with DBS punches in wells;
Add 100 μL of elution buffer to each well containing a DBS punch to elute sample from the DBS punch;
Add 75 ng Eu-labelled detection antibody, in this example, the anti-NT-proBNP detAb was mouse anti-human NT-proBNP clone 13G12 from HyTest, to each well;
The plate was then incubated for 180 min;
The punch and liquid were then removed from the wells of the plate and each well was washed 6 times;
Then, 200 μL of inducer solution was added. The inducer solution creates a fluorescent Europium or Samarium chelate for measuring DELFIA time-resolved fluorescence (TRF) assays. In this example, the inducer solution includes Triton X-1001, hydrochloric acid, and chelators;
The mixture is then incubated for 5 min with shaking followed by measurement of time-resolved fluorescence of Europium released from the detection antibody.

In this example, sST2 and Galectin-3 were assayed using a 2-step assay as follows:
Take an anti-sST2 or anti-Galectin-3 capture antibody-coated 96-well plate with DBS punches in wells;
Add 100 μL of elution buffer to each well containing a DBS punch to elute sample from the DBS punch; incubate 60 min with shaking;
Wash 4 times;
Add 75 ng Eu-labelled detection antibody, in this example the anti-sSt2 detection antibody was mouse anti-human sST2 monoclonal antibody Clone 10206 SPTN-5 from Medix, or mouse anti-human Galectin-3 Clone 10304 SPTN-5 from Medix, in assay buffer to each well;
The plate or plates were then incubated for 30 min with shaking;
The punch and liquid were then removed from the wells of the plate and each well was washed 4 times;
Then, 200 μL of inducer solution was added. The inducer solution creates a fluorescent Europium or Samarium chelate for measuring DELFIA time-resolved fluorescence (TRF) assays. In this example, the inducer solution includes Triton X-1001, hydrochloric acid, and chelators;
The mixture is then incubated for 5 min with shaking followed by measurement of time-resolved fluorescence of Europium released from the detection antibody.

DBS calibrators used in this example were created by spiking recombinant (or native) antigen into washed red blood cells in known concentrations e.g. 0, 1, 3, 10, 30, 100 ng/ml or 0, 0.1, 0.3, 1.0, 3.0, 10 ng/mL, in general to cover the clinically relevant concentration ranges, are also included in the plate and unknown sample signal level is compared to those signals coming from the calibrators.

Data analysis values obtained from these assays for NT-proBNP, sST2, and Galectin-3 were compared between CHD and non-CHD samples. Group medians, averages and distributions were compared. Levels were normalized with known underlying co-factors, such as birth weight, Gestational age (GA) week of delivery, sex of newborn, and day of testing after birth, for possible bias effects. Risk assessment was done with sample concentration, normalized or not, using a single or multiple cut-off values. Then the combination of biomarkers was evaluated using binary, linear, and non-linear models with or without co-factor normalization. An algorithm to estimate overall risk was applied to all samples in this example in order to separate CHD and non-CHD samples, then POX (pulseoximetry) was used as a pre-screen to remove those picked up with it and used biochemical markers, i.e. NT-proBNP, sST2, and Galectin-3, to separate remaining CHD from non-CHD cases.

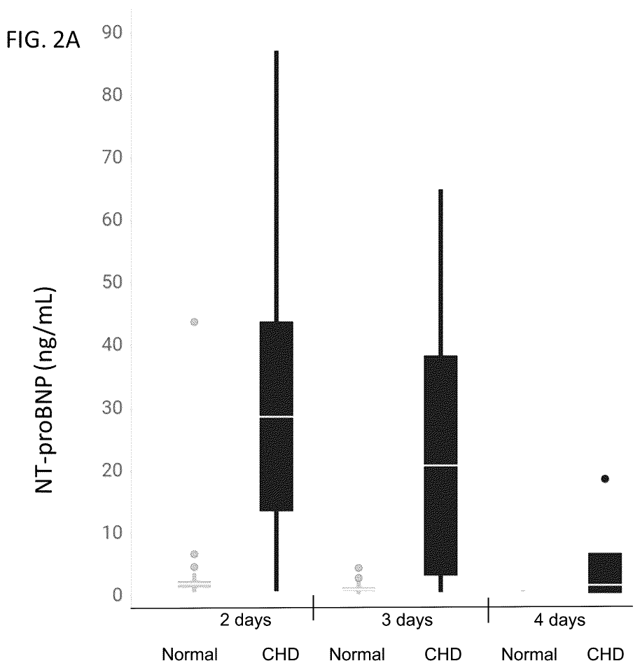

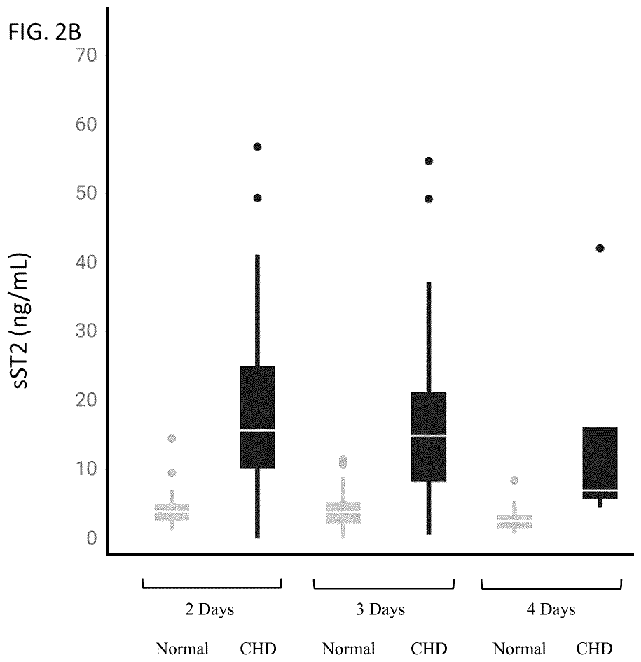

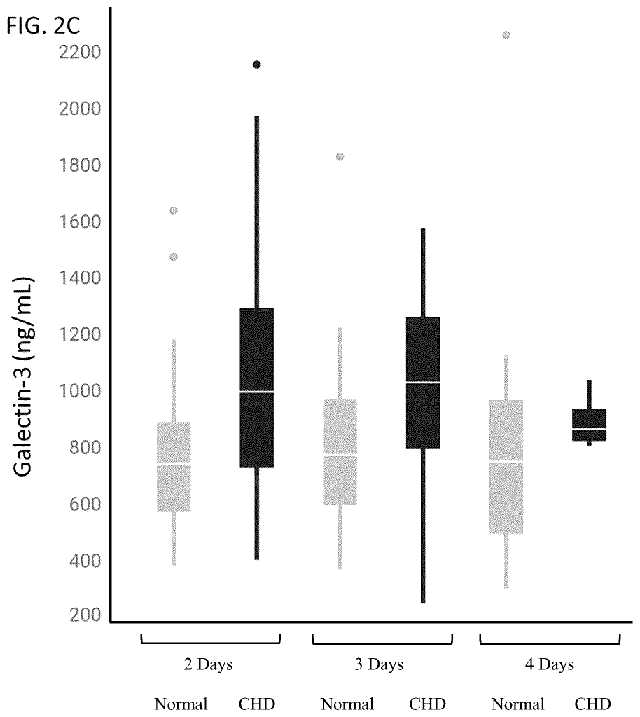

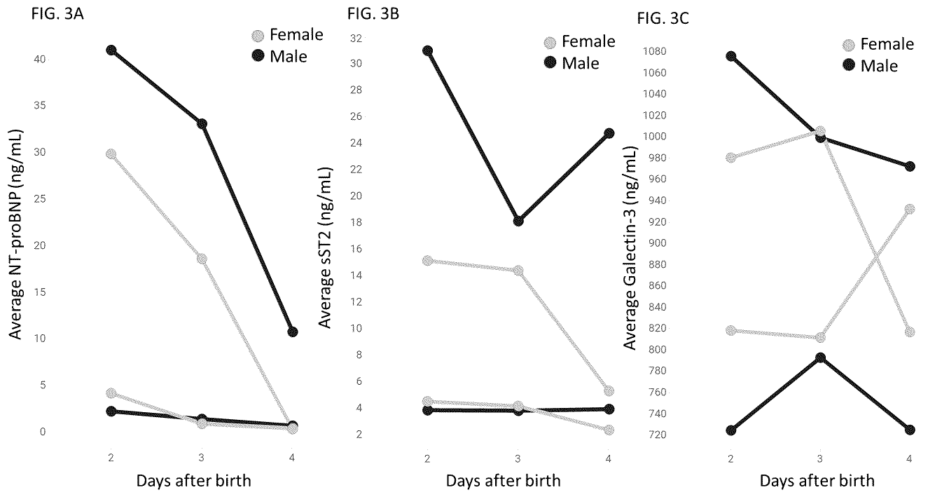

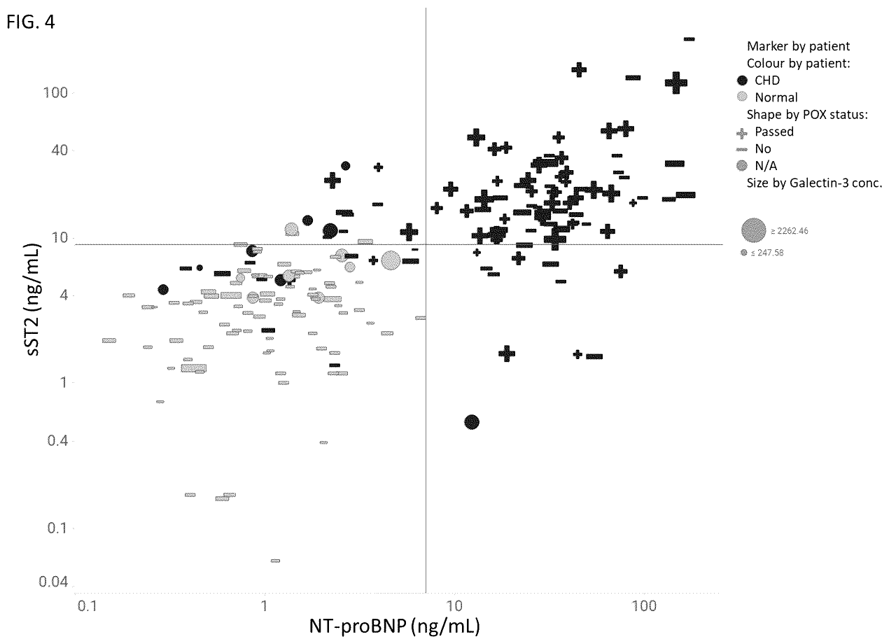

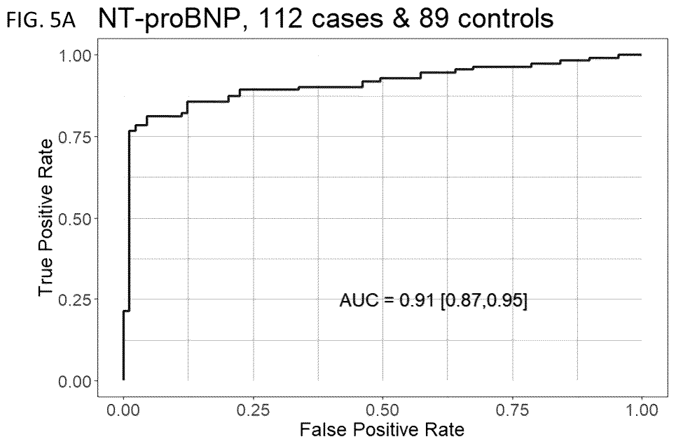

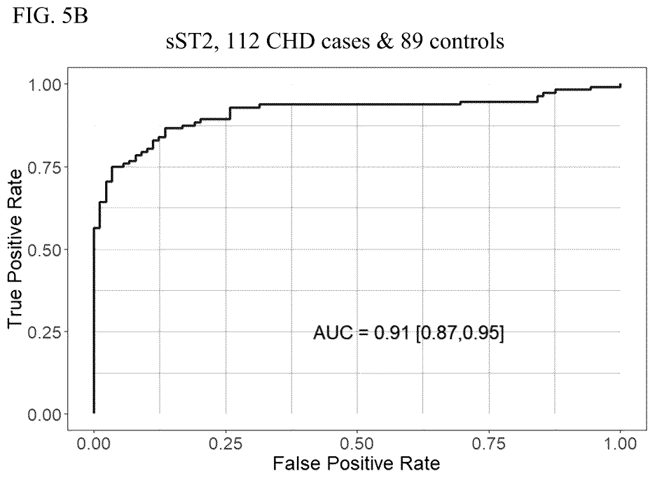

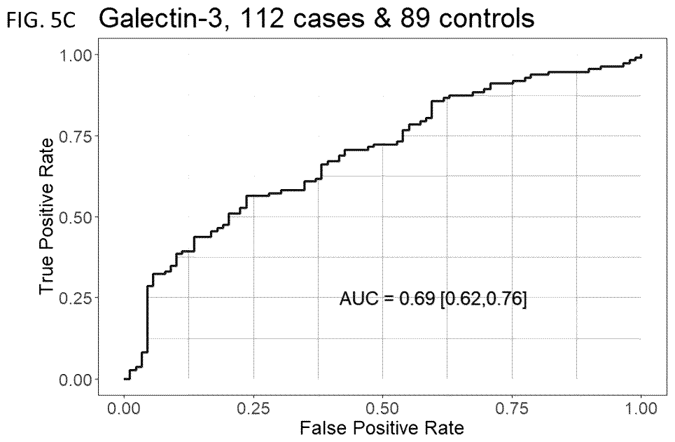

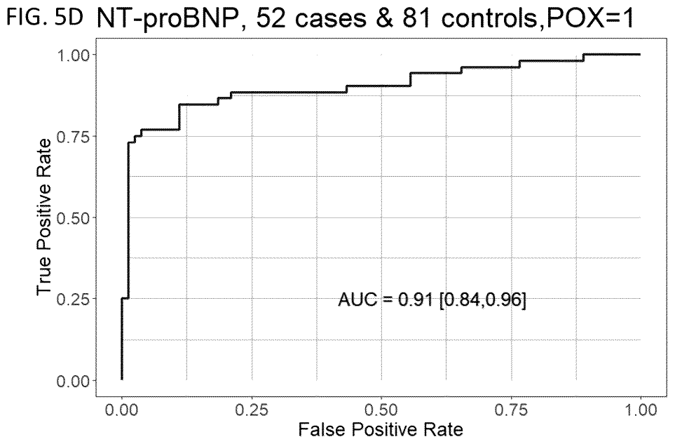

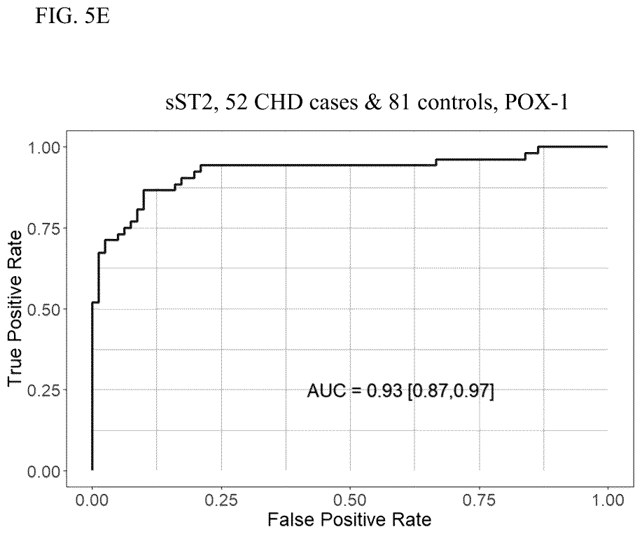

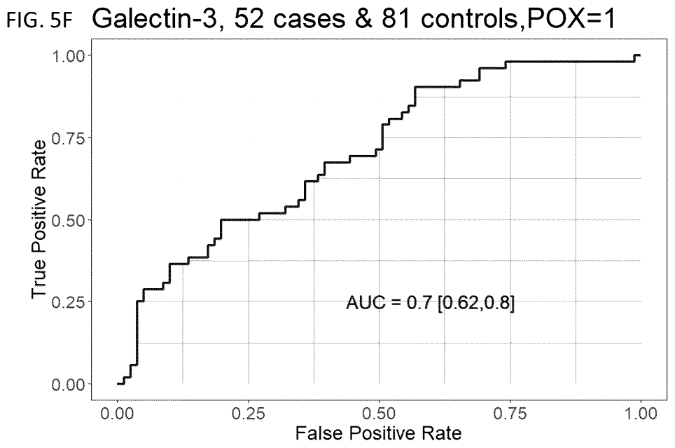

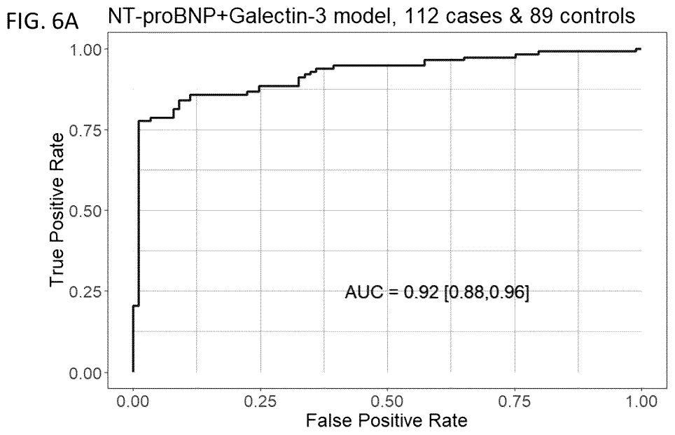

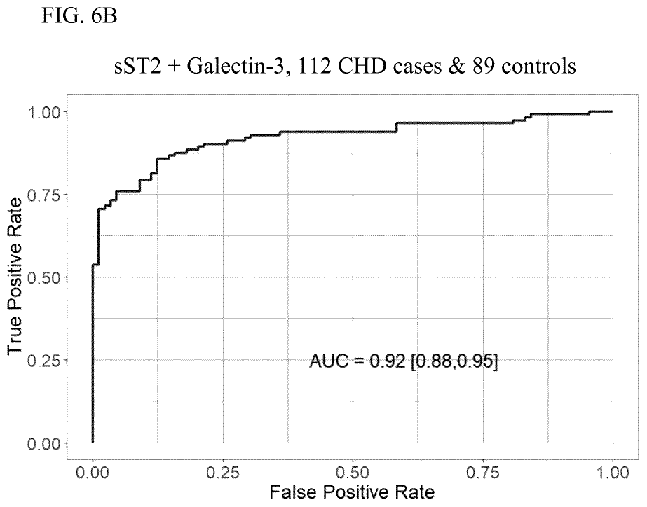

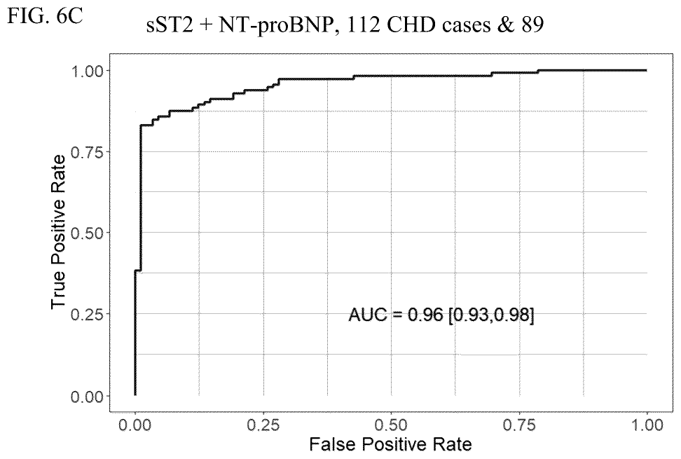

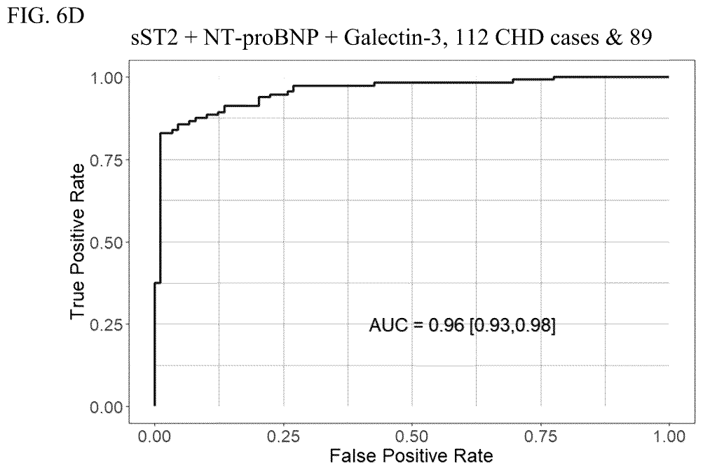

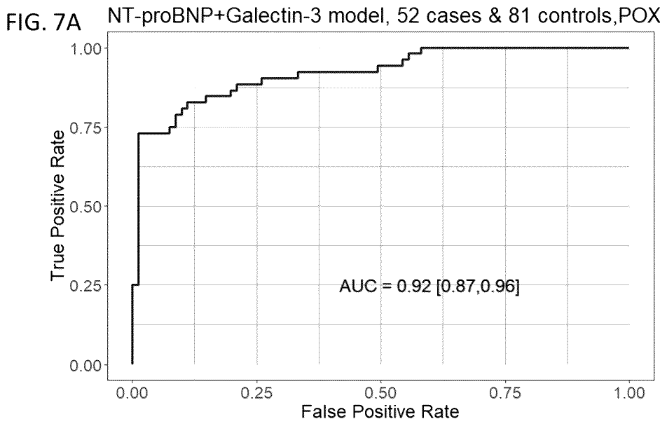

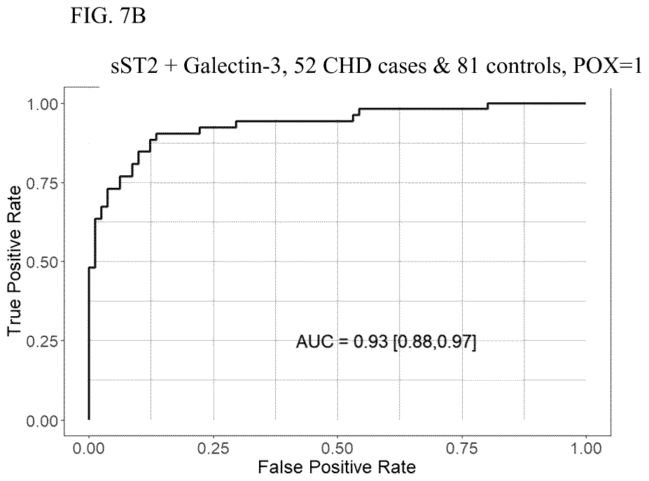

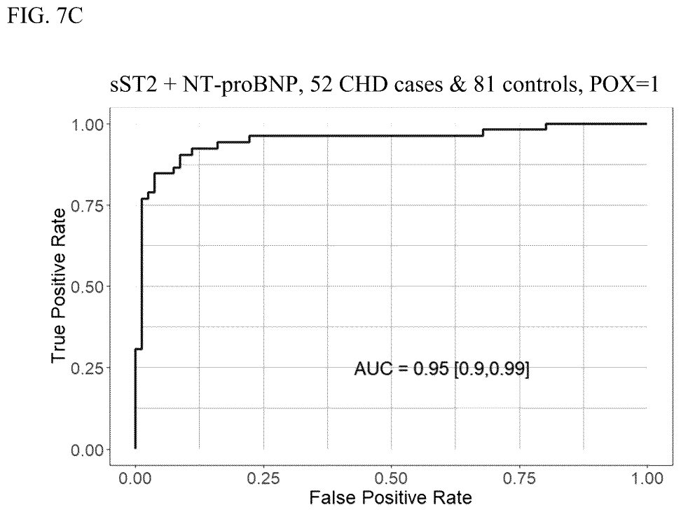

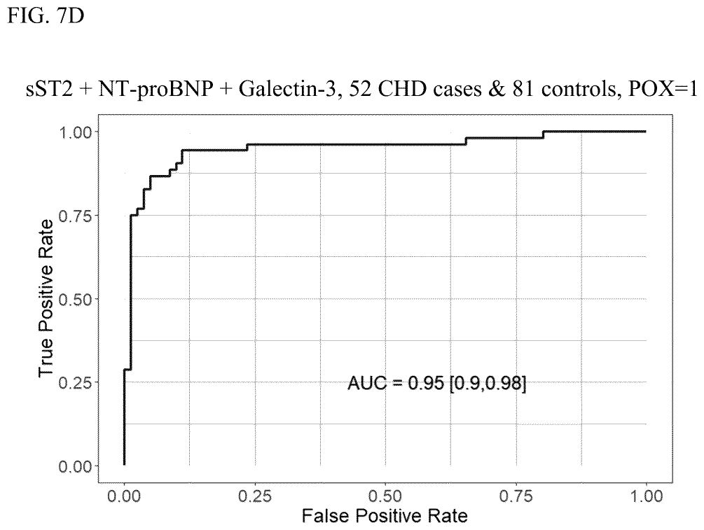

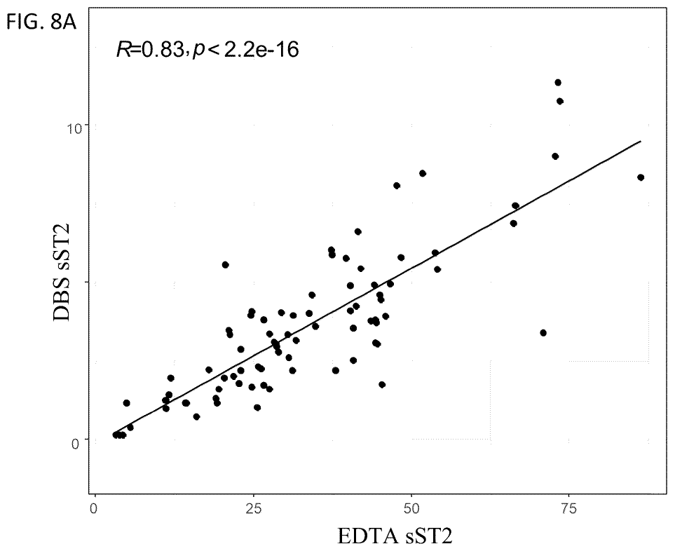

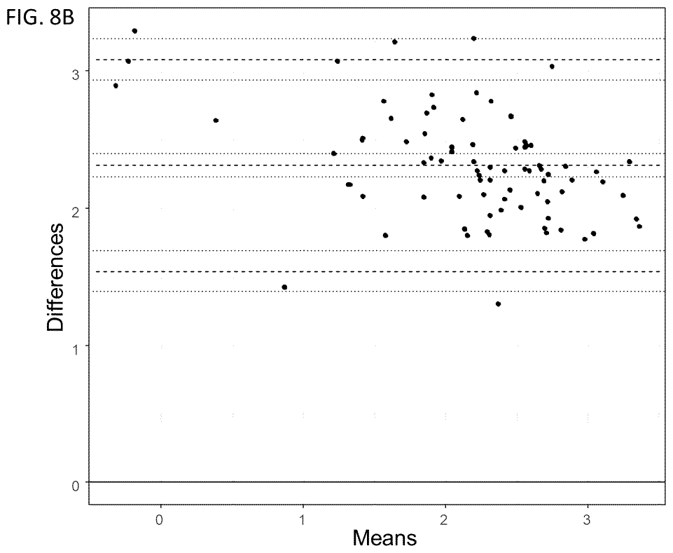

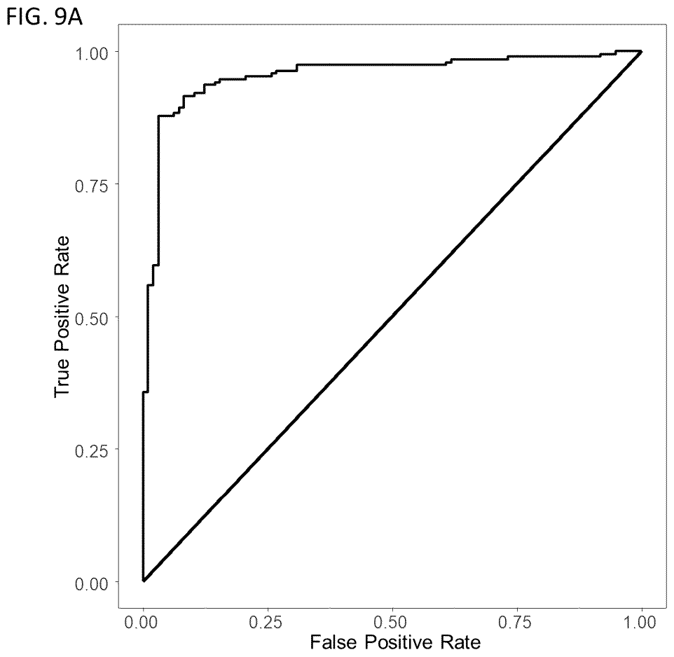

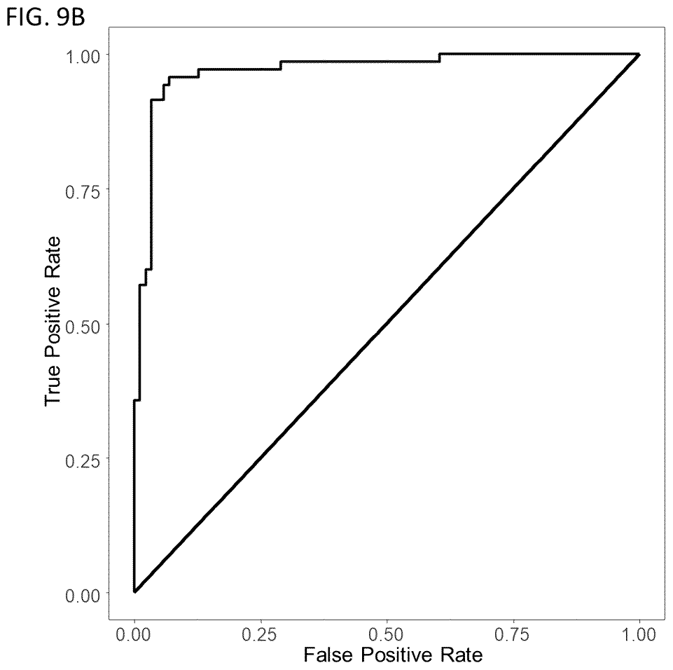

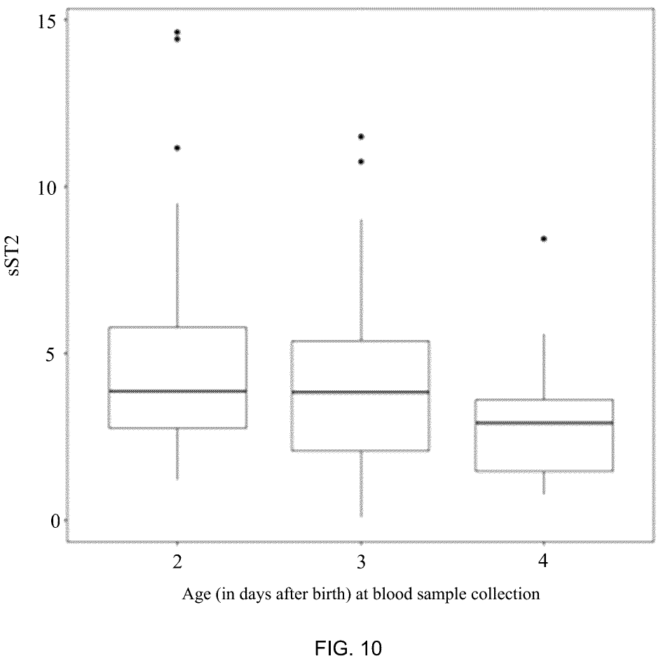

Results of assays and analyses described herein are shown in FIGS. 2A, 2B, 2C, 3A, 3B, 3C, 4, 5A, 5B, 5C, 5D, 5E, 5F, 6A, 6B, 6C, 6D, 7A, 7B, 7C, 7D, 8A, 8B, 9A, 9B, and 10.

### Items

Item 1. A method of detection of congenital heart defect (CHD) in a newborn human patient, comprising: performing an assay for one or more biomarkers of CHD selected from the group consisting of: soluble Suppression of Tumorigenicity 2 protein (sST2), and Galectin-3 in a dried blood sample obtained from the newborn human patient, thereby determining a level of the one or more biomarkers present in the dried blood sample; and comparing the determined level of the one or more biomarkers present in the dried blood sample with a standard representing a normal newborn control without CHD, thereby determining whether the determined level is increased or decreased compared to the standard, wherein an increased level of the one or more biomarkers present in the dried blood sample compared with a standard representing a normal newborn control without congenital heart defect indicates detection of CHD in the newborn human patient.

Item 2. The method of item 1, further comprising an assay for N-terminal pro b-type natriuretic peptide (NT-proBNP) as a biomarker of CHD.

Item 3. The method of item 1 or item 2, wherein the assay is an assay for: 1) sST2 and NT-proBNP; 2) sST2 and Galectin-3; 3) NT-proBNP and Galectin-3, or 4) all of sST2, NT-proBNP and Galectin-3.

Item 4. The method of any of items 1, 2, or 3, wherein the assay comprises an immunoassay.

Item 5. The method of any of items 1 to 4, wherein a blood sample is obtained from the newborn human patient: on the day of birth, one day after birth, two days after birth, three days after birth, four days after birth, five days after birth, six days after birth, one week after birth, or later.

Item 6. The method of any of items 1 to 5 further comprising assessment of the newborn human patient using pulse oximetry (POX).

Item 7. The method of any of items 1 to 6, further comprising assessment of one or more physical characteristics of the newborn human patient.

Item 8. The method of any of items 1 to 7, further comprising assessment of one or more physiological characteristics of the newborn human patient.

Item 9. A kit for detection of CHD in a newborn human patient, comprising: one or more reagents for performing an assay for one or more biomarkers of CHD selected from the group consisting of: soluble Suppression of Tumorigenicity 2 protein (sST2), and Galectin-3 in a dried blood sample obtained from the newborn human patient, to determine a level of the one or more biomarkers present in the dried blood sample.

Item 10. The kit of item 9, wherein the one or more reagents comprises one or more antibodies specific for sST2, and/or Galectin-3.

Item 11. The kit of item 10, further comprising one or more antibodies specific for N-terminal pro b-type natriuretic peptide (NT-proBNP),

Item 12. The kit of any of items 9, 10, or 11, further comprising one or more reagents or devices for one or more of: collection, drying, transport, and storage, of a blood sample from a newborn human patient.

Item 13. A computer-implemented method of detection of CHD in a newborn human patient, comprising: providing a determined level of one or more biomarkers selected from the group consisting of: sST2, and Galectin-3 present in a dried blood sample of the newborn human patient; responsive to the determined level, assigning a score to the newborn human patient representative of detection of CHD by algorithmically comparing the determined level(s) to a standard or standards representing one or more normal newborn controls without congenital heart defect, thereby detecting congenital heart defect in the newborn human patient.

Item 14. The computer-implemented method of detection of congenital heart defect in a newborn human patient according to item 13, further comprising providing a determined level of NT-proBNP.

Item 15. The computer-implemented method of detection of congenital heart defect in a newborn human patient according to item 13 or item 14, further comprising providing one or more types of data selected from the group consisting of: pulse oximetry data from the newborn human patient, one or more physical characteristics of the newborn human patient, and one or more physiological characteristics of the newborn human patient producing a normalized determined level of one or more of: sST2, Galectin-3, and NT-proBNP normalized with respect to the one or more types of data.

Item 16. The computer-implemented method of detection of congenital heart defect in a newborn human patient according to item 15, comprising algorithmically comparing the normalized determined level of the one or more of: sST2, Galectin-3, and NT-proBNP with a standard or standards representing one or more normal newborn controls without congenital heart defect normalized with respect to the one or more types of data.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of detection of congenital heart defect (CHD) in a newborn human patient, comprising:
performing an assay for one or more biomarkers of CHD selected from the group consisting of: soluble Suppression of Tumorigenicity 2 protein (sST2), and Galectin-3 in a dried blood sample obtained from the newborn human patient, thereby determining a level of the one or more biomarkers present in the dried blood sample;
comparing the determined level of the one or more biomarkers present in the dried blood sample with a standard representing a normal newborn control without CHD, thereby determining whether the determined level is increased or decreased compared to the standard, wherein an increased level of the one or more biomarkers present in the dried blood sample compared with a standard representing a normal newborn control without congenital heart defect indicates detection of CHD in the newborn human patient.

2. The method of claim 1, further comprising an assay for N-terminal pro b-type natriuretic peptide (NT-proBNP) as a biomarker of CHD.

3. The method of claim 1, wherein the assay is an assay for: 1) sST2 and NT-proBNP; 2) sST2 and Galectin-3; 3) NT-proBNP and Galectin-3, or 4) all of SST2, NT-proBNP and Galectin-3.

4. The method of claim 1, wherein the assay comprises an immunoassay.

5. The method of claim 1, wherein a blood sample is obtained from the newborn human patient: on the day of birth, one day after birth, two days after birth, three days after birth, four days after birth, five days after birth, six days after birth, one week after birth, or later.

6. The method of claim 1 further comprising assessment of the newborn human patient using pulse oximetry (POX).

7. The method of claim 1, further comprising assessment of one or more physical characteristics of the newborn human patient.

8. The method of claim 1, further comprising assessment of one or more physiological characteristics of the newborn human patient.

9. A kit for detection of CHD in a newborn human patient, comprising: one or more reagents for performing an assay for one or more biomarkers of CHD selected from the group consisting of: soluble Suppression of Tumorigenicity 2 protein (sST2), and Galectin-3 in a dried blood sample obtained from the newborn human patient, to determine a level of the one or more biomarkers present in the dried blood sample.

10. The kit of claim 9, wherein the one or more reagents comprises one or more antibodies specific for sST2, and/or Galectin-3.

11. The kit of claim 10, further comprising one or more antibodies specific for N-terminal pro b-type natriuretic peptide (NT-proBNP).

12. The kit of claim 9, further comprising one or more reagents or devices for one or more of: collection, drying, transport, and storage, of a blood sample from a newborn human patient.

13. A computer-implemented method of detection of CHD in a newborn human patient, comprising:
providing a determined level of one or more biomarkers selected from the group consisting of: sST2, and Galectin-3 present in a dried blood sample of the newborn human patient; responsive to the determined level, assigning a score to the newborn human patient representative of detection of CHD by algorithmically comparing the determined level(s) to a standard or standards representing one or more normal newborn controls without congenital heart defect, thereby detecting congenital heart defect in the newborn human patient.

14. The computer-implemented method of detection of congenital heart defect in a newborn human patient according to claim 13, further comprising providing a determined level of NT-proBNP.

15. The computer-implemented method of detection of congenital heart defect in a newborn human patient according to claim 13, further comprising providing one or more types of data selected from the group consisting of: pulse oximetry data from the newborn human patient, one or more physical characteristics of the newborn human patient, and one or more physiological characteristics of the newborn human patient producing a normalized determined level of one or more of: sST2, Galectin-3, and NT-proBNP normalized with respect to the one or more types of data.

16. The computer-implemented method of detection of congenital heart defect in a newborn human patient according to claim 15, comprising algorithmically comparing the normalized determined level of the one or more of: sST2, Galectin-3, and NT-proBNP with a standard or standards representing one or more normal newborn controls without congenital heart defect normalized with respect to the one or more types of data.
